(19) United States
(12) Patent Application Publication
(10) Pub. No.: US 2005/0171616 A1
(43) Pub. Date: Aug. 4, 2005

(54) Peritoneal regeneration with acellular pericardial patch

(76) Inventors: Hsing-Wen Sung, HsinChu (TW); Po-Hong Lai, Taoyuan County (TW); Huang-Chien Liang, Hsinchu (TW); Hosheng Tu, Newport Beach, CA (US)

Correspondence Address: HOSHENG TU, 15 RIEZ, NEWPORT BEACH, CA, 92657-0116, US

(21) Appl. No.: 11/053,053

(22) Filed: Feb. 8, 2005

Related U.S. Application Data
(63) Continuation-in-part of application No. 10/408,176, filed on Apr. 7, 2003.
Application No. 10/408,176 is a continuation-in-part of application No. 10/067,130, filed on Feb. 4, 2002, now Pat. No. 6,545,042.
(60) Provisional application No. 60/544,612, filed on Feb. 13, 2004.

(51) Int. Cl. A61F 2/02; A61F 2/04

(52) U.S. Cl. 623/23.72; 623/23.71

(57) ABSTRACT

The invention discloses a method of using acellular bovine pericardia fixed with genipin as a surgical-repair material to fix an abdominal wall defect.

### CROSS-REFERENCE TO RELATED APPLICATIONS

This patent application is a continuation-in-part application of U.S. patent application Ser. No. 10/408,176, filed Mar. 7, 2003, which is a continuation-in-part application of application Ser. No. 10/067,130, filed Feb. 4, 2002, now U.S. Pat. No. 6,545,042. The application is related to U.S. patent application Ser. No. 10/717,162, filed Nov. 19, 2003, Ser. No. 10/610,391 filed Jun. 30, 2003, and Ser. No. 10/211,656 filed Aug. 2, 2002, now U.S. Pat. No. 6,624,138, This application also claims priority benefits of provisional application Ser. No. 60/544,612, filed Feb. 13, 2004. Entire contents of all above co-pending applications are incorporated herein by reference.

### FIELD OF THE INVENTION

The present invention generally relates to chemical modification of biomedical materials, such as collagen matrix with a naturally occurring crosslinking reagent, genipin. More particularly, the present invention relates to crosslinkable biological solution as medical material prepared with bioactive agents and the crosslinking reagent, genipin, its derivatives or analog and the process thereof.

### BACKGROUND OF THE INVENTION

Crosslinking of biological molecules is often desired for optimum effectiveness in biomedical applications. For example, collagen, which constitutes the structural framework of biological tissue, has been extensively used for manufacturing bioprostheses and other implanted structures, such as vascular grafts, wherein it provides a good medium for cell infiltration and proliferation. However, biomaterials derived from collagenous tissue must be chemically modified and subsequently sterilized before they can be implanted in humans. The fixation, or crosslinking, of collagenous tissue increases strength and reduces antigenicity and immunogenicity.

Collagen sheets are also used as wound dressings, providing the advantages of high permeability to water vapor and rapid wound healing. Disadvantages include low tensile strength and easy degradation of collagen by collagenase. Crosslinking of collagen sheets reduces cleavage by collagenase and improves tensile strength.

Clinically, biological tissue has been used in manufacturing heart valve prostheses, small-diameter vascular grafts, and biological patches, among others. However, the biological tissue has to be fixed with a crosslinking or chemically modifying agent and subsequently sterilized before they can be implanted in humans. The fixation of biological tissue is to reduce antigenicity and immunogenicity and prevent enzymatic degradation. Various crosslinking agents have been used in fixing biological tissue. These crosslinking agents are mostly synthetic chemicals such as formaldehyde, glutaraldehyde, dialdehyde starch, glyceraldehydes, cyanamide, diimides, diisocyanates, and epoxy compound. However, these chemicals are all highly cytotoxic which may impair the biocompatibility of biological tissue. Of these, glutaraldehyde is known to have allergenic properties, causing occupational dermatitis and is cytotoxic at concentrations greater than 10-25 ppm and as low as 3 ppm in tissue culture. It is therefore desirable to provide a crosslinking agent suitable for use in biomedical applications that is within acceptable cytotoxicity and that forms stable and biocompatible crosslinked products.

To achieve this goal, a naturally occurring crosslinking agent (genipin) has been used to fix biological tissue or crosslinkable biological solution. The co-pending application Ser. No. 09/297,808 filed Sep. 27, 2001, entitled “Chemical modification of biomedical materials with genipin” is incorporated and cited herein by reference. The cytotoxicity of genipin was previously studied in vitro using 3T3 fibroblasts, indicating that genipin is substantially less cytotoxic than glutaraldehyde (Sung H W et al., J Biomater Sci Polymer Edn 1999;10:63-78). Additionally, the genotoxicity of genipin was tested in vitro using Chinese hamster ovary (CHO-K1) cells, suggesting that genipin does not cause clastogenic response in CHO-K1 cells (Tsai C C et al., J Biomed Mater Res 2000;52:58-65). A biological material treated with genipin resulting in acceptable cytotoxicity is key to biomedical applications.

It is further hypothesized in the literature that acellular tissue might remove cellular antigens (Wilson G J et al., Trans Am Soc Artif Intern 1990;36:340-343). As a means for reducing the antigenic response to xenograft material, cell extraction removes lipid membranes and membrane-associated antigens as well as soluble proteins. Courtman et al. developed a cell extraction process to render bovine pericardium free of cells and soluble proteins, leaving a framework of largely insoluble collagen and elastin (Courtman D W et al., J Biomed Mater Res 1994;28:655-666). They hypothesized that this process may decrease the antigenic load within the material, reducing the associated degradation due to in vivo cellular attack, and possibly eliminating the need for extensive crosslinking. Additionally, acellular tissue may provide a natural microenvironment for host cell migration to accelerate tissue regeneration (Malone J M et al., J Vasc Surg 1984;1:181-91).

Other than maintaining a natural microenvironment, the collagen matrix, including soluble collagen, after being treated with the proposed cell extraction process, the collagen matrix shall have similar properties of decreased antigenicity/immunogenicity. However, the framework of largely insoluble collagen and elastin matrix is still vulnerable to enzymatic degradation and is not suitable as an implantable bioprosthesis.

As is well known that the human knee comprises an articulation of the femur, the tibia and the patella. The femur and the tibia are maintained in a condition of stable articulation by a number of ligaments of which the principal ones are the anterior and posterior cruciate ligaments and the collateral ligaments. The rupture of the anterior cruciate ligament is relatively commonly encountered as a result of sporting injury or the like. This rupture leads to knee instability and can be a debilitating injury. Though less common, the rupture of the posterior cruciate ligament can be equally disabling.

In the past, polymer or plastic materials have been studied as ligament or tendon replacements. Prosthetic ligament replacements made of carbon fibers and Gore-Tex PTFE materials do not last a long period of time. Repeated loading of a prosthetic ligament in a young active patient leads to failure of the ligament. It has been found that it is difficult to provide a tough durable plastic material which is suitable for long-term connective tissue replacement. Plastic material could become infected and difficulties in treating such infections often lead to graft failure.

In accordance with the present invention, there is provided genipin treated tissue grafts for orthopedic and other surgical applications, such as vascular grafts and heart valve bioprostheses, which have shown to exhibit many of the desired characteristics important for optimal graft function. In particular, the tissue regeneration capability in the genipin-fixed acellular tissue may be suitable as a graft material for bone, tendon, ligament, cartilage, muscle, and cardiovascular applications.

In some aspects of the invention, it is provided a method for promoting autogenous ingrowth of a biological tissue material, comprising providing a natural tissue, removing cellular material from the natural tissue, increasing porosity of the natural tissue by at least 5%, loading an angiogenesis agent or autologous cells into the porosity, and crosslinking the natural tissue with a crosslinking agent.

Some aspects of the invention relate to crosslinkable biological solution configured and adapted for promoting angiogenesis, wherein the crosslinkable biological solution is incorporated with an organic angiogenic agent such as ginsenoside Rg₁, ginsenoside Re or the like.

### SUMMARY OF THE INVENTION

In general, it is an object of the present invention to provide a biological scaffold configured and adapted for tissue regeneration or tissue engineering. In one embodiment, the process of preparing a biological scaffold comprises steps of removing cellular material from a natural tissue and crosslinking the natural tissue with genipin, wherein the scaffold is characterized by reduced antigenicity, reduced immunogenicity and reduced enzymatic degradation upon placement inside a patient's body. The “tissue engineering” in this invention may include cell seeding, cell ingrowth and cell proliferation into the scaffold or collagen matrix in vivo or in vitro.

It is another object of the present invention to provide a tendon or ligament graft for use as connective tissue substitute, wherein the graft is formed from a segment of connective tissue protein, and the segment is crosslinked with genipin, its analog or derivatives resulting in reasonably acceptable cytotoxicity and reduced enzymatic degradation.

It is a further object of the present invention to provide a method for promoting autogenous ingrowth of damaged or diseased tissue selected from a group consisting of bone, ligaments, tendons, muscle and cartilage, the method comprising a step of surgically repairing the damaged or diseased tissue by attachment of a tissue graft, wherein the graft is formed from a segment of connective tissue protein, the segment being crosslinked with genipin, its analog or derivatives with acceptable cytotoxicity and reduced enzymatic degradation, and wherein the tissue graft is loaded with growth factors or bioactive agents.

In some aspects, there is provided a biological tissue material or tissue sheet material configured and adapted for tissue regeneration comprising steps of removing cellular material from a natural tissue and crosslinking the natural tissue with a crosslinking agent or with ultraviolet irradiation, the tissue material being characterized by reduced antigenicity, reduced immunogenicity and reduced enzymatic degradation upon placement inside a patient's body, wherein porosity of the natural tissue is increased by at least 5%, the increase of porosity being adapted for promoting tissue regeneration. In a preferred embodiment, the tissue material is selected from a group consisting of a tissue valve, a tissue valve leaflet, a vascular graft, a ureter, a urinary bladder, a dermal graft, and the like. In another preferred embodiment, the natural tissue or tissue sheet material is selected from a group consisting of a porcine valve, a bovine jugular vein, a bovine pericardium, an equine pericardium, a porcine pericardium, an ovine pericardium, a valvular leaflet, submucosal tissue, and the like. In still another embodiment, the crosslinked acellular natural tissue material is loaded with at least one growth factor or at least one bioactive agent.

In some aspects, there is provided a method for promoting autogenous ingrowth of a biological tissue material comprising the steps of providing a natural tissue, removing cellular material from the natural tissue, increasing porosity of the natural tissue by at least 5%, and crosslinking the natural tissue with a crosslinking agent. The tissue material is generally characterized by reduced antigenicity, reduced immunogenicity and reduced enzymatic degradation upon placement inside a patient's body. In one embodiment, the crosslinked acellular natural tissue is loaded with growth factors or bioactive agents.

In some aspects, there is provided a method for promoting autogenous ingrowth of a biological tissue material comprising the steps of providing a natural tissue, removing cellular material from the natural tissue, increasing porosity of the natural tissue by at least 5%, loading an angiogenesis agent or autologous cells into the porosity, and crosslinking the natural tissue with a crosslinking agent. In one preferred embodiment, the angiogenesis agent is ginsenoside Rg₁, ginsenoside Re, or selected from the group consisting of VEGF, VEGF 2, bFGF, VEGF121, VEGF165, VEGF189, VEGF206, PDGF, PDAF, TGF-β, PDEGF, PDWHF, and combination thereof.

Some aspects of the invention relate to a method for promoting angiogenesis for treating tissue comprising: providing crosslinkable biological solution to the target tissue, wherein the crosslinkable biological solution may be loaded with at least one angiogenic agent (also known as angiogenic growth factor) or bioactive agent. In one embodiment, the at least one angiogenic agent is a protein agent selected from a group consisting of VEGF, VEGF 2, bFGF, VEGF121, VEGF165, VEGF189, VEGF206, PDGF, PDAF, TGF-β, PDEGF, PDWHF, and combination thereof. In a preferred embodiment, the at least one angiogenic agent is an organic agent selected from a group consisting of ginsenoside Rg₁, ginsenoside Re, combination thereof and the like. In another embodiment, the crosslinkable biological solution of the present invention is broadly defined in a form or phase of solution, paste, gel, suspension, colloid or plasma that may be solidifiable thereafter. In still another embodiment, the crosslinkable biological solution of the invention is crosslinkable with a crosslinking agent or with ultraviolet irradiation before, during or after the step of tissue treatment.

Some aspects of the invention relate to a drug-collagen-genipin and/or drug-chitosan-genipin compound that is loadable onto an implant or stent enabling drug slow-release to the surrounding tissue, or to the lumen of the bodily cavity. In one preferred embodiment, the compound is loaded onto the outer periphery of the stent enabling drug slow-release to the surrounding tissue.

It is another object of the present invention to provide a crosslinkable biological solution kit comprising a first readily mixable crosslinkable biological solution component and a second crosslinker component, wherein an operator can add appropriate drugs or bioactive agents to the kit and obtain a drug-collagen-genipin and/or drug-chitosan-genipin compound enabling drug slow-release to the target tissue. In a further embodiment, the crosslinkable biological solution kit is packaged in a form for topical administration, for percutaneous injection, for intravenous injection, for intramuscular injection, for loading on an implant or biological tissue material, and/or for oral administration.

Some aspects of the invention relate to a method of repairing abdominal wall defects, comprising patching the defects with acellular bovine pericardium fixed with genipin enabling successfully preventing the formation of postsurgical abdominal adhesions.

Some aspects of the invention relate to a method of repairing a tissue or organ defect in a patient, comprising: providing an acellular tissue sheet material having mechanical strengths; repairing the defect by appropriately placing the tissue material at the defect; and allowing tissue regeneration into the tissue material. In a further embodiment, the tissue sheet material is selected from a group consisting of a bovine pericardium, an equine pericardium, an ovine pericardium, a porcine pericardium, and a valvular leaflet. In another embodiment, the tissue sheet material is crosslinked with a crosslinking agent or with ultraviolet irradiation, wherein the crosslinking agent may be selected from a group consisting of genipin, its analog, derivatives, and combination thereof, aglycon geniposidic acid, epoxy compounds, dialdehyde starch, glutaraldehyde, formaldehyde, dimethyl suberimidate, carbodiimides, succinimidyls, diisocyanates, acyl azide, reuterin, and combination thereof.

The method of repairing a tissue or organ defect in a patient further comprises a process of increasing porosity of the acellular tissue sheet material, the process being selected from a group consisting of an enzyme treatment process, an acid treatment process, and a base treatment process, wherein the increase of porosity of the tissue material is 5% or higher. In one embodiment, the defect is an abdominal wall defect, a vascular wall defect, a valvular leaflet defect, a heart tissue defect, or the like.

In some embodiments, the tissue material of the invention further comprises at least one growth factor selected from a group consisting of vascular endothelial growth factor, transforming growth factor-beta, insulin-like growth factor, platelet derived growth factor, fibroblast growth factor, and combination thereof. In one embodiment, the tissue material comprises ginsenoside Rg₁, ginsenoside Re, or at least one bioactive agent.

Some aspects of the invention relate to a method of treating postsurgical tissue or organ adhesion comprising: providing an acellular tissue sheet material; placing the acellular tissue sheet material around or about the tissue or organ to be treated; and preventing the tissue sheet material from forming the postsurgical adhesion, wherein the adhesion may be abdominal adhesion. In one further embodiment, the tissue sheet material is crosslinked with a crosslinking agent or with ultraviolet irradiation, wherein the crosslinking agent is selected from a group consisting of genipin, its analog, derivatives, and combination thereof, aglycon geniposidic acid, epoxy compounds, dialdehyde starch, glutaraldehyde, formaldehyde, dimethyl suberimidate, carbodiimides, succinimidyls, diisocyanates, acyl azide, reuterin, and combination thereof.

Some aspects of the invention relate to a method of treating postsurgical tissue or organ adhesion comprising topically administering an anti-adhesion solution at about the tissue or organ of the surgical site, wherein the solution comprises a crosslinkable biological solution and a crosslinking agent, wherein the crosslinking agent may be selected from a group consisting of genipin, its analog, derivatives, and combination thereof, aglycon geniposidic acid, epoxy compounds, dialdehyde starch, glutaraldehyde, formaldehyde, dimethyl suberimidate, carbodiimides, succinimidyls, diisocyanates, acyl azide, reuterin, and combination thereof In a further embodiment, the anti-adhesion solution further comprises at least one growth factor selected from a group consisting of vascular endothelial growth factor, transforming growth factor-beta, insulin-like growth factor, platelet derived growth factor, fibroblast growth factor, ginsenoside Rg₁ growth factor and ginsenoside Re growth factor.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 is chemical structures of glutaraldehyde and genipin that are used in the chemical treatment examples of the current disclosure.

FIG. 2 are photomicrographs of H&E stained tissue for (a) specimen-A, cellular tissue; (b) specimen-B, acellular tissue; (c) specimen-C, the acid treated acellular tissue; and (d) specimen-D, the enzyme treated acellular tissue.

FIG. 3 shows the SEM of bovine pericardia tissue for (a) specimen-A, cellular tissue; (b) specimen-B, acellular tissue; (c) specimen-C, the acid treated acellular tissue; and (d) specimen-D, the enzyme treated acellular tissue.

FIG. 4 shows porosity of bovine pericardia tissue for (a) specimen-A, cellular tissue; (b) specimen-B, acellular tissue; (c) specimen-C, the acid treated acellular tissue; and (d) specimen-D, the enzyme treated acellular tissue.

FIG. 5 shows thickness of the glutaraldehyde-fixed cellular tissue (A/GA), the glutaraldehyde-fixed acellular tissue (B/GA), the genipin-fixed cellular tissue (A/GP), and the genipin-fixed acellular tissue (B/GP) before implantation.

FIG. 6 show denaturation temperature values of the non-crosslinked and genipin-crosslinked bovine pericardia tissue for (a) specimen-A, cellular tissue; (b) specimen-B, acellular tissue; (c) specimen-C, the acid treated acellular tissue; and (d) specimen-D, the enzyme treated acellular tissue.

FIG. 7 shows thickness of the bovine pericardia tissue before and after genipin crosslinking for (a) specimen-A, cellular tissue; (b) specimen-B, acellular tissue; (c) specimen-C, the acid treated acellular tissue; and (d) specimen-D, the enzyme treated acellular tissue.

FIG. 8 are photomicrographs of H&E stained genipin-crosslinked tissue for (a) specimen-A/GP, cellular tissue; (b) specimen-B/GP, acellular tissue; (c) specimen-C/GP, the acid treated acellular tissue; and (d) specimen-D/GP, the enzyme treated acellular tissue retrieved at 3-day postoperatively.

FIG. 9 are cells infiltration extents of genipin-crosslinked bovine pericardia tissue for (a) specimen-A/GP, cellular tissue; (b) specimen-B/GP, acellular tissue, (c) specimen-C/GP, the acid treated acellular tissue; and (d) specimen-D/GP, the enzyme treated acellular tissue retrieved at 3 days and 4-week postoperatively.

FIG. 10 are tensile-strength values of the glutaraldehyde-fixed cellular tissue (A/GA), the glutaraldehyde-fixed acellular tissue (B/GA), the genipin-fixed cellular tissue (A/GP), and the genipin-fixed acellular tissue (B/GP) before implantation and those retrieved at several distinct duration of post implantation.

FIG. 11 is an illustration of the suggested mechanism of tissue regeneration in the outer layers of the acellular tissue as disclosed in the present invention wherein B/GA denotes the glutaraldehyde-fixed acellular tissue and B/GP denotes the genipin-fixed acellular tissue.

FIG. 12 is a chemical formula of ginsenoside Rg₁.

FIG. 13 are cells infiltration extents of genipin-crosslinked acellular bovine pericardia tissue with angiogenesis factors for (a) specimen-AGP, without Rg₁; (b) light microscopy of specimen a; (c) specimen-AGP, with Rg₁; and (d) light microscopy of specimen c; all explants retrieved at 1-week postoperatively.

FIG. 14 is an animal myocardial patch study design for myocardial tissue regeneration.

FIG. 15 is 4-week postoperative results on animal myocardial patch study of FIG. 14: photomicrographs of Masson Trichrome stained tissue.

FIG. 16 is 4-week postoperative results on animal myocardial patch study of FIG. 14: photomicrographs of Factor VIII stained tissue.

FIG. 17 is a chemical formula for Ginsenoside Re.

FIG. 18 is a preparation method of loading an acellular tissue with growth factors Rg₁, Re, or BFGF.

FIG. 19 is 1-week postoperative results on animal angiogenesis study: photomicrographs of H&E (hematoxylin and eosin) stained tissue.

FIG. 20 is 1-week postoperative results on animal angiogenesis study, photomicrographs of SEM tissue.

FIG. 21 is 1-week postoperative results on animal angiogenesis study: quantification of neo-capillaries and tissue hemoglobin.

FIG. 22A is an iridoid glycoside present in fruits of Gardenia jasmindides Ellis (Structure I).

FIG. 22B is a parent compound geniposide (Structure II) from which genipin is derived.

FIG. 23 is a crosslinkable biological solution kit comprising a first crosslinkable biological solution component and a second crosslinker component.

FIG. 24 show photographs of the implanted polypropylene mesh (Polypropylene) and the AGA, GP, and AGP patches. AGA: the glutaraldehyde-fixed acellular tissue; GP: the genipin-fixed cellular tissue; AGP: the genipin-fixed acellular tissue.

FIG. 25 show representative photographs for each studied group retrieved at 1-month and 3-month postoperatively. Polypropylene: the polypropylene mesh; AGA: the glutaraldehyde-fixed acellular tissue; GP: the genipin-fixed cellular tissue; AGP: the genipin-fixed acellular tissue.

FIG. 26 show photomicrographs of the polypropylene mesh (Polypropylene) and the AGA, GP, and AGP patches retrieved at 1-month postoperatively stained with H&E (200× magnification). AGA: the glutaraldehyde-fixed acellular tissue; GP: the genipin-fixed cellular tissue; AGP: the genipin-fixed acellular tissue.

FIG. 27 show photomicrographs of the polypropylene mesh (Polypropylene) and the AGA, GP, and AGP patches retrieved at 3-month postoperatively stained with H&E (200× magnification). AGA: the glutaraldehyde-fixed acellular tissue; GP: the genipin-fixed cellular tissue; AGP: the genipin-fixed acellular tissue.

FIG. 28 show photomicrographs of the polypropylene mesh (Polypropylene) and the AGA, GP, and AGP patches retrieved at 3-month postoperatively obtained by the immunohistochemical stain (800× magnification). AGA: the glutaraldehyde-fixed acellular tissue; GP: the genipin-fixed cellular tissue; AGP: the genipin-fixed acellular tissue.

FIG. 29 show photomicrographs of the AGP patch retrieved at 3-month postoperatively obtained by the immunohistochemical stains to identify neo-collagen type I and III and those retrieved at 1-month and 3-month postoperatively stained with van Gieson to identify mesothelial cells (800× magnification).

FIG. 30 show fracture-tension values of the polypropylene mesh (Polypropylene) and the AGA, GP, and AGP patches before implantation and those retrieved at distinct implantation durations. AGA: the glutaraldehyde-fixed acellular tissue; GP: the genipin-fixed cellular tissue; AGP: the genipin-fixed acellular tissue.

FIG. 31 show calcium contents of the polypropylene mesh (Polypropylene) and the AGA, GP, and AGP patches retrieved at distinct implantation durations. AGA: the glutaraldehyde-fixed acellular tissue; GP: the genipin-fixed cellular tissue; AGP: the genipin-fixed acellular tissue.

FIG. 32 show adhesion scores for the polypropylene mesh (Polypropylene), the glutaraldehyde-fixed acellular tissue (AGA), the genipin-fixed cellular tissue (GP), and the genipin-fixed acellular tissue (AGP) retrieved at distinct durations postoperatively.

### DETAILED DESCRIPTION OF EXEMPLARY EMBODIMENTS

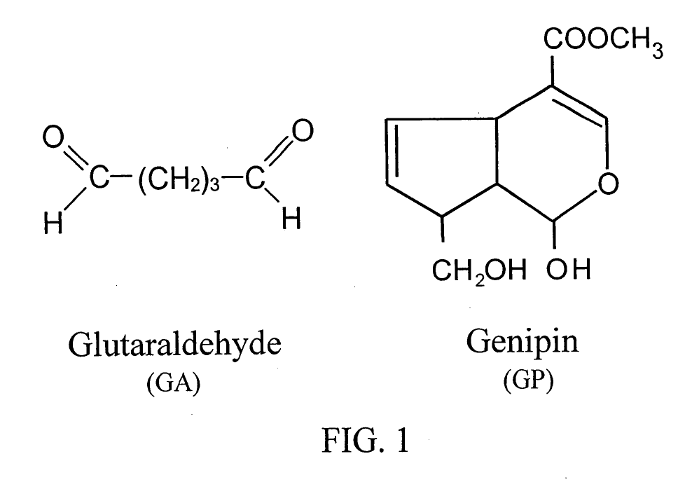

The following detailed description, with accompanied FIG. 1 to FIG. 32, is of the best presently contemplated modes of carrying out the invention. This description is not to be taken in a limiting sense, but is made merely for the purpose of illustrating general principles of embodiments of the invention.

“Genipin” in this invention is meant to refer to the naturally occurring compound as shown in FIG. 1 and its derivatives, analog, stereoisomers and mixtures thereof.

“Tissue engineering” or “tissue regeneration” in meant to refer to cell seeding, cell ingrowth and cell proliferation into the acellular scaffold or collagen matrix in vivo or in vitro, sometimes enhanced with an angiogenesis factor.

A “biological tissue material” refers to a biomedical material or device of biological tissue origin which is inserted into, or grafted onto, bodily tissue to remain for a period of time, such as an extended-release drug delivery device, tissue valve, tissue valve leaflet, vascular or dermal graft, ureter, urinary bladder, or orthopedic prosthesis, such as bone, ligament, tendon, cartilage, and muscle.

“Crosslinkable biological solution” is herein meant to refer to collagen extract, soluble collagen, elastin, gelatin, chitosan, N, O, carboxylmethyl chitosan (NOCC), chitosan-containing and other collagen-containing biological solution. For a preferred aspect of the present invention, the biological solution is meant to indicate a crosslinkable biological substrate that may comprise at least a genipin-crosslinkable functional group, such as amino group or the like, or crosslinkable with UV irradiation. The crosslinkable biological solution of the present invention is broadly defined in a form or phase of solution, paste, gel, suspension, colloid or plasma that may be solidifiable thereafter.

An “implant” refers to a medical device (of biological and non-biological origin) which is inserted into, or grafted onto, bodily tissue to remain for a period of time, such as an extended-release drug delivery device, tissue valve, tissue valve leaflet, drug-eluting stent, vascular graft, wound healing or skin graft, orthopedic prosthesis, such as bone, ligament, tendon, cartilage, and muscle.

A “scaffold” in this invention is meant to refer to a tissue matrix substantially or completely devoid of cellular materials. A scaffold may further comprise added structure porosity for cell ingrowth or proliferation.

An “acellularization process” is meant to indicate the process for removing at least a portion of cells from cellular tissue and/or tissue matrix containing connective tissue protein.

“Drug” in this invention is meant to broadly refer to a chemical molecule(s), biological molecule(s) or bioactive agent providing a therapeutic, diagnostic, or prophylactic effect in vivo. “Drug” and “bioactive agent” (interchangeable in meaning) may comprise, but not limited to, synthetic chemicals, biotechnology-derived molecules, herbs, cells, genes, growth factors, health food and/or alternate medicines. In the present invention, the terms “drug” and “bioactive agent” are sometimes used interchangeably.

It is one object of the present invention to provide an acellular biological scaffold chemically treated with a naturally occurring crosslinking agent, genipin, that is configured and adapted for tissue regeneration, and/or tissue engineering in biomedical applications. In a region with suitable substrate diffusivity, an acellular biological tissue material with added porosity and chemically treated by a crosslinking agent enables tissue regeneration, and/or tissue engineering in many biomedical applications.

Preparation and Properties of Genipin

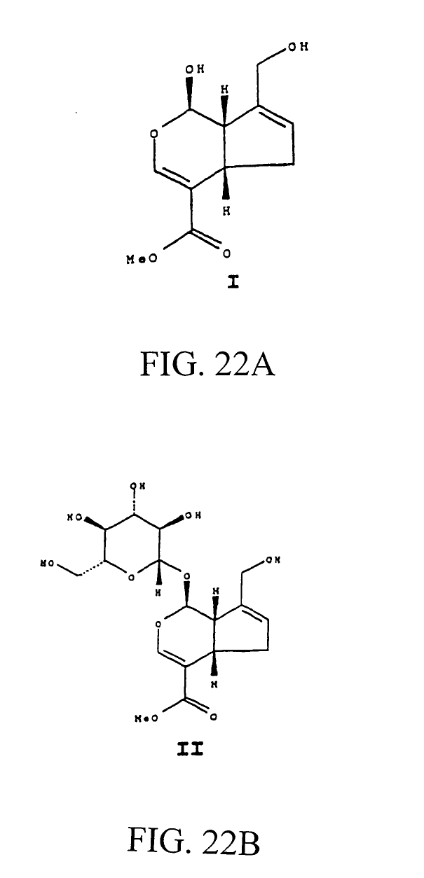

Genipin, shown in Structure I of FIG. 22A, is an iridoid glycoside present in fruits (Gardenia jasmindides Ellis). It may be obtained from the parent compound geniposide, Structure II (FIG. 22B), which may be isolated from natural sources as described in elsewhere. Genipin, the aglycone of geniposide, may be prepared from the latter by oxidation followed by reduction and hydrolysis or by enzymatic hydrolysis. Alternatively, racemic genipin may be prepared synthetically. Although Structure I shows the natural configuration of genipin, any stereoisomer or mixture of stereoisomers of genipin as shown later may be used as a crosslinking reagent, in accordance with the present invention.

Genipin has a low acute toxicity, with LD₅₀ i.v. 382 mg/kg in mice. It is therefore much less toxic than glutaraldehyde and many other commonly used synthetic crosslinking reagents. As described below, genipin is shown to be an effective crosslinking agent for treatment of biological materials intended for in vivo biomedical applications, such as prostheses and other implants, wound dressings, and substitutes.

The genipin derivatives and/or genipin analog may have the following chemical formulas (Formula 1 to Formula 4): 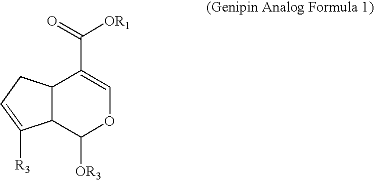
in which
R₁ represents lower alkyl;

R₂ represents lower alkyl, pyridylcarbonyl, benzyl or benzoyl;

R₃ represents formyl, hydroxymethyl, azidomethyl, 1-hydroxyethyl, acetyl, methyl, hydroxy, pyridylcarbonyl, cyclopropyl, aminomethyl substituted or unsubstituted by (1,3-benzodioxolan-5-yl)carbonyl or 3,4,5-trimethoxybenzoyl, 1,3-benzodioxolan-5-yl, ureidomethyl substituted or unsubstituted by 3,4,5-trimethoxyphenyl or 2-chloro-6-methyl-3-pyridyl, thiomethyl substituted or unsubstituted by acetyl or 2-acetylamino2-ethoxycarbonyethyl, oxymethyl substituted or unsubstituted by benzoyl, pyridylcarbonyl or 3,4,5-trimethoxybenzoyl;
provided that R₃ is not methyl formyl, hydroxymethyl, acetyl, methylaminomethyl, acetylthiomethyl, benzoyloxymethyl or pyridylcarbonyloxymethyl when R₁ is methyl, and
its pharmaceutically acceptable salts, or stereoisomers. 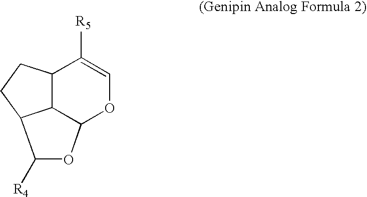
in which
R₄ represents lower alkoxy, benzyloxy, benzoyloxy, phenylthio, C₁˜C₁₂ alkanyloxy substituted or unsubstituted by t-butyl, phenyl, phenoxy, pyridyl or thienyl;

R₅ represents methoxycarbonyl, formyl, hydroxyiminomethyl, methoxyimino-methyl, hydroxymethyl, phenylthiomethyl or acetylthiomethyl;
provided that R₅ is not methoxycarbonyl when R₁₄ is acetyloxy; and
its pharmaceutically acceptable salts, or stereoisomers. 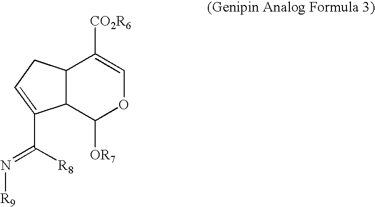
R₆ represents hydrogen atom, lower alkyl or alkalimetal;
R₇ represents lower alkyl or benzyl;
R₈ represents hydrogen atom or lower alkyl;
R₉ represents hydroxy, lower alkoxy, benzyloxy, nicotinoyloxy, isonicotinoyloxy, 2-pyridylmethoxy or hydroxycarbonylmethoxy;
provided that R₉ is not hydroxy or methoxy when R₆ is methyl and R₈ is hydrogen atom; and
its pharmaceutically acceptable salts, or stereoisomers. 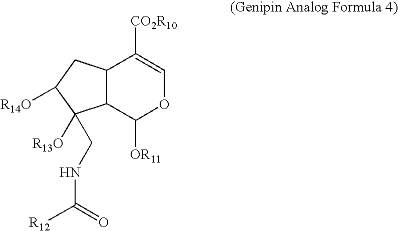
in which
R₁₀ represents lower alkyl;
R₁₁ represents lower alkyl or benzyl;
R₁₂ represents lower alkyl, pyridyl substituted or unsubstituted by halogen, pyridylamino substituted or unsubstituted by lower alkyl or halogen, 1,3-benzodioxolanyl;
R₁₃ and R₁₄ each independently represent a hydrogen atom or join together to form isopropylidene; and
its pharmaceutically acceptable salts, or stereoisomers.

Kyogoku et al. in U.S. Pat. No. 5,037,664, U.S. Pat. No. 5,270,446, and EP 0366998, entire contents of all three being incorporated herein by reference, teach the crosslinking of amino group containing compounds with genipin and the crosslinking of genipin with chitosan. They also teach the crosslinking of iridoid compounds with proteins which can be vegetable, animal (collagen, gelatin) or microbial origin. However, they do not teach loading drug onto a collagen-containing biological material or solution crosslinked with genipin as biocompatible drug carriers for drug slow-release.

Smith in U.S. Pat. No. 5,322,935, incorporated herein by reference in its entirety, teaches the crosslinking of chitosan polymers and then further crosslinking again with covalent crosslinking agents like glutaraldehyde. Smith, however, does not teach loading drug onto a chitosan-containing biological material crosslinked with genipin as biocompatible drug carriers for drug slow-release.

Previously, Chang in U.S. Pat. No. 5,929,038 discloses a method for treating hepatitis B viral infection with an iridoid compound of a general formula containing a six-member hydrocarbon ring sharing with one common bondage of a five-member hydrocarbon ring. Further, Moon et al. in U.S. Pat. No. 6,162,826 and No. 6,262,083 discloses genipin derivatives having anti hepatitis B virus activity and liver protection activity. All of which three aforementioned patents are incorporated herein by reference. The teachings of these patents do not disclose preparing tissue/device with scaffolds or collagen matrix with desirable porosity for use in tissue engineering, wherein the raw material source for tissue engineering is chemically modified by genipin, genipin derivatives or its analog with acceptably minimal cytotoxicity.

Noishiki et al. in U.S. Pat. No. 4,806,595 discloses a tissue treatment method by a crosslinking agent, polyepoxy compounds. Collagens used in that patent include an insoluble collagen, a soluble collagen, an atelocollagen prepared by removing telopeptides on the collagen molecule terminus using protease other than collagenase, a chemically modified collagen obtained by succinylation or esterification of above-described collagens, a collagen derivative such as gelatin, a polypeptide obtained by hydrolysis of collagen, and a natural collagen present in natural tissue (ureter, blood vessel, pericardium, heart valve, etc.) The Noishiki et al. patent is incorporated herein by reference. “Collagen matrix” in the present invention is collectively used referring to the above-mentioned collagens, collagen species, collagen in natural tissue, and collagen in a biological implant preform.

Voytik-Harbin et al. in U.S. Pat. No. 6,264,992 discloses submucosa as a growth substrate for cells. More particularly, the submucosa is enzymatically digested and gelled to form a shape retaining gel matrix suitable for inducing cell proliferation and growth both in vivo and in vitro. The Voytik-Harbin et al. patent is incorporated herein by reference. Collagen matrix chemically modified or treated by genipin of the present invention may serve as a shapeable raw material for making a biological implant preform adapted for inducing cell proliferation and ingrowth, but also resisting enzymatic degradation, both in vivo and in vitro.

Cook et al. in U.S. Pat. No. 6,206,931 discloses a graft prosthesis material including a purified, collagen-based matrix structure removed from a submucosa tissue source, wherein the submucosa tissue source is purified by disinfection and removal steps to deactivate and remove contaminants. The Cook et al. patent is incorporated herein by reference. Similarly, a collagen-based matrix structure, also known as “collagen matrix” in this disclosure, may serve as a biomaterial adapted for medical device use after chemical modification by genipin of the present invention.

Levene et al. in U.S. Pat. No. 6,103,255 discloses a porous polymer scaffold for tissue engineering, whereby the scaffold is characterized by a substantially continuous solid phase, having a highly interconnected bimodal distribution of open pore sizes. The Levene et al. patent is incorporated herein by reference. The present invention discloses biological scaffolds by acellular process and acidic/enzymatic treatment adapted for tissue engineering. Additional benefits of genipin tissue treatment for reduced antigenicity, reduced cytotoxicity and enhanced biodurability are disclosed in the present invention.

Bell in U.S. Pat. No. 6,051,750, No. 5,893,888, and No. 5,800,537 discloses method and construct for producing graft tissue from extracellular matrix, wherein the matrix particulates are seeded with living human cells or fused to constitute composites of various shape. The Bell patents are incorporated herein by reference. A collagen matrix with genipin treatment of the present invention enables a building material to constitute composites of various shapes, sizes of a medical prosthesis or biological implants.

In one embodiment, the crosslinker or crosslinking agent of the invention may be selected from a group consisting of genipin, its analog, derivatives, and combination thereof, aglycon geniposidic acid, epoxy compounds, dialdehyde starch, glutaraldehyde, formaldehyde, dimethyl suberimidate, carbodiimides, succinimidyls, diisocyanates, acyl azide, reuterin, tris(hydroxymethyl)phosphine, ascorbate-copper, glucose-lysine, and combination thereof. A co-pending U.S. patent application Ser. No. 10/924,539, filed Aug. 24, 2004 and Ser. No. 10/929,047, filed Aug. 27, 2004, entire contents of both are incorporated herein by reference, disclose medical use of reuterin and aglycon geniposidic acid as crosslinking agents.

### EXAMPLE 1

### Tissue Specimen Preparation

In one embodiment of the present invention, bovine pericardia procured from a slaughterhouse are used as raw materials. The procured pericardia are transported to the laboratory in a cold normal saline. In the laboratory, the pericardia are first gently rinsed with fresh saline to remove excess blood on tissue. Adherent fat is then carefully trimmed from the pericardial surface. The cleaned/trimmed pericardium before acellular process is herein coded specimen-A. The procedure used to remove the cellular components from bovine pericardia is adapted from a method developed by Courtman et al (J Biomed Mater Res 1994;28:655-66), which is also referred to herein as “an acellularization process”. A portion of the trimmed pericardia is then immersed in a hypotonic tris buffer (pH 8.0) containing a protease inhibitor (phenylmethyl-sulfonyl fluoride, 0.35 mg/L) for 24 hours at 4° C. under constant stirring. Subsequently, they are immersed in a 1% solution of Triton X-100 (octylphenoxypolyethoxyethanol; Sigma Chemical, St. Louis, Mo., USA) in tris-buffered salt solution with protease inhibition for 24 hours at 4° C. under constant stirring. Samples then are thoroughly rinsed in Hanks' physiological solution and digested with DNase and RNase at 37° C. for 1 hour. This is followed by a further 24-hour extraction with Triton X-100 in tris buffer. Finally, all samples are washed for 48 hours in Hanks' solution and the acellular sample is coded specimen-B. Light microscopic examination of histological sections from extracted tissue revealed an intact connective tissue matrix with no evidence of cells.

A portion of the acellular tissue of bovine pericardia (specimen-B) is further treated with 1% acetic acid at room temperature for one hour. The acidic component is thereafter removed from the tissue by lyophilization at about −50° C. for 24 hours, followed by thorough rinse with filtered water to obtain the acellular pericardia having enlarged pore or added porosity. The tissue is stored in phosphate buffered saline (PBS, 0.01M, pH 7.4, Sigma Chemical), which tissue is coded specimen-C. The procedure of acetic acid treatment to add porosity is referred herein as “acid treatment”. Similar results could be achieved by following the acid treatment with other diluted acid solution, such as nitric acid or the like, at the comparable acidity or pH vales.

The mechanism of increasing the tissue porosity treated by a mild acidic solution lies in the effect of [H⁺] or [OH⁻] values on the collagen fibers matrix of the acellular tissue. It is postulated and disclosed that acellular tissue treated with a base solution (i.e., a solution pH value greater than 7.0) could have the same effect upon enlarged pores or added porosity.

A portion of the bovine pericardia tissue post-acid treatment (i.e., specimen-C) is further treated with enzymatic collagenase as follows. Add 0.01 gram of collagenase to a beaker of 40 ml TES buffer and incubate the specimen-C pericardia tissue at 37° C. for 3 hours. The sample is further treated with 10 mM EDTA solution, followed by thorough rinse. The tissue is stored in phosphate buffered saline (PBS, 0.01M, pH 7.4, Sigma Chemical), which tissue is coded specimen-D. The procedure of collagenase treatment to add porosity is referred herein as “enzyme treatment”.

EXAMPLE 2

### Tissue Specimen Crosslinking

The cellular tissue (specimen-A) and acellular tissue (specimen-B) of bovine pericardia are fixed in 0.625% aqueous glutaraldehyde (Merck KGaA, Darmstadt, Germany) and are coded as specimen-A/GA and specimen-B/GA, respectively. Furthermore, the cellular tissue (specimen-A) and acellular tissue (specimen-B, specimen-C, and specimen-D) of bovine pericardia are fixed in genipin (Challenge Bioproducts, Taiwan) solution at 37° C. for 3 days and are coded as specimen-A/GP, specimen-B/GP, specimen-C/GP, and specimen-D/GP, respectively. The aqueous glutaraldehyde and genipin solutions used are buffered with PBS. The amount of solution used in each fixation was approximately 200 mL for a 10×10 cm bovine pericardium. After fixation, the thickness of each studied group is determined using a micrometer (Digimatic Micrometer MDC-25P, Mitutoyo, Tokyo, Japan). Subsequently, the fixed cellular and acellular tissue are sterilized in a graded series of ethanol solutions with a gradual increase in concentration from 20 to 75% over a period of 4 hours. Finally, the test tissue is thoroughly rinsed in sterilized PBS for approximately 1 day, with solution change several times, and prepared for tissue characterization as well as a subcutaneous study. The chemical structures of the crosslinking agents (genipin and glutaraldehyde as control) used in the study are shown in FIG. 1.

In the present invention, the terms “crosslinking”, “fixation”, “chemical modification”, and/or “chemical treatment” for tissue or biological solution are used interchangeably.

Though the methods for removing cells from cellular tissue and/or acid treatment, base treatment, enzyme treatment to enlarge pores are well known to those who are skilled in the art, it is one object of the present invention to provide an acellular biological scaffold chemically treated with a naturally occurring crosslinking agent, genipin, that is configured and adapted for tissue regeneration, and/or tissue engineering in biomedical applications with acceptable cytotoxicity and reduced enzymatic degradation.

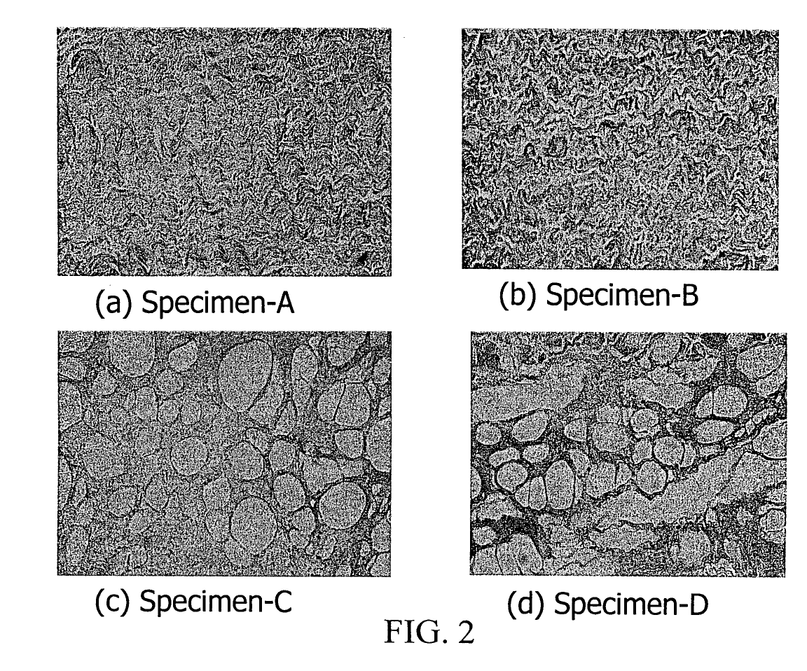

FIG. 2 shows photomicrographs of H&E (hematoxylin and eosin) stained tissue for (a) specimen-A, cellular tissue; (b) specimen-B, acellular tissue; (c) specimen-C, the acid treated acellular tissue; and (d) specimen-D, the enzyme treated acellular tissue. As shown in FIG. 2(a), the bovine pericardia prior to cell extraction shows a number of intact cells with identifiable cell nuclei embedded within the connective tissue matrices. In contrast, the extracted tissue revealed an intact connective tissue matrix with no evidence of cells (FIGS. 2(b)-2(d)). Some open spaces within the acellular tissue are apparent with acid treated specimen-C and enzyme treated specimen-D.

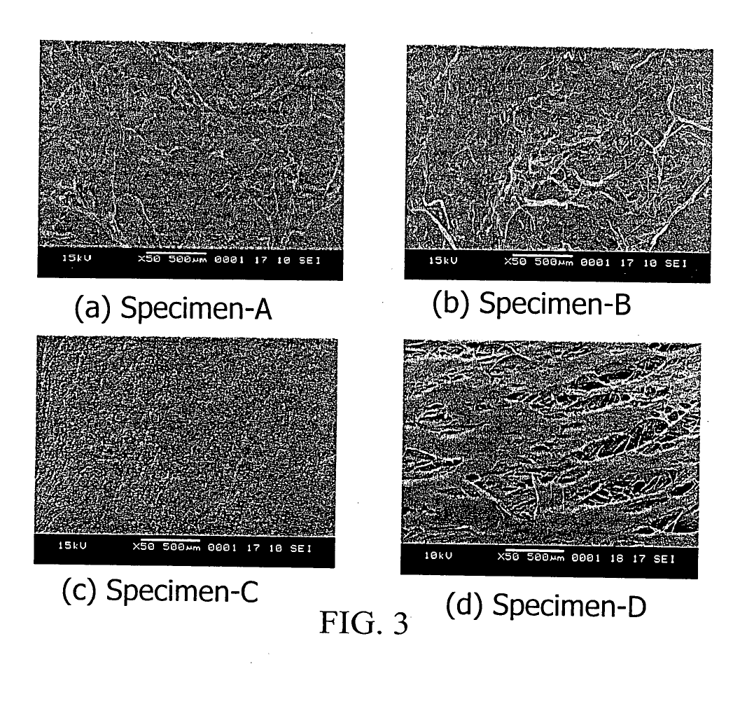

FIG. 3 shows the SEM (scanning electron microscopy) of bovine pericardia tissue for (a) specimen-A, cellular tissue; (b) specimen-B, acellular tissue; (c) specimen-C, the acid treated acellular tissue; and (d) specimen-D, the enzyme treated acellular tissue. The enzyme treated specimen-D shows several enlarged pores up to a couple of hundred microns, which would serve as a scaffold for enhanced tissue infiltration in tissue engineering.

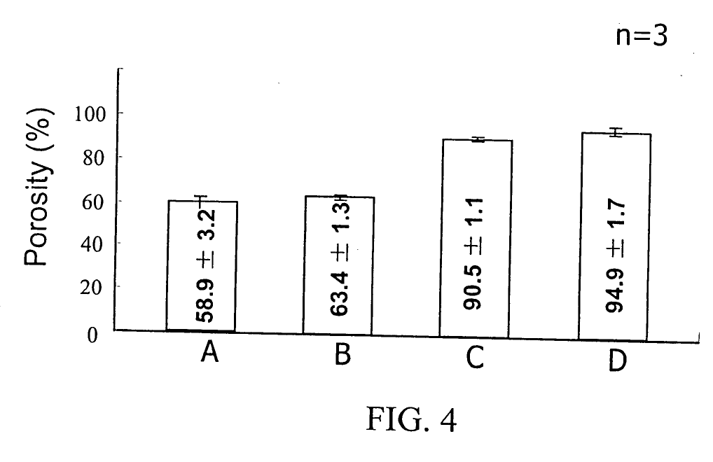

FIG. 4 shows porosity of bovine pericardia tissue for (a) specimen-A, cellular tissue; (b) specimen-B, acellular tissue; (c) specimen-C, the acid treated acellular tissue; and (d) specimen-D, the enzyme treated acellular tissue. “Porosity” is defined as the fraction of the void over the total apparent volume. The overall porosity of the acid treated and enzyme treated acellular tissue is substantial higher than the control cellular tissue. It is suggested that a tissue scaffold of the specimen-C or specimen-D type is desirable in tissue engineering applications for tissue infiltration or cells ingrowth.

### EXAMPLE 3

### Comparison of Glutaraldehyde and Genipin Crosslinking

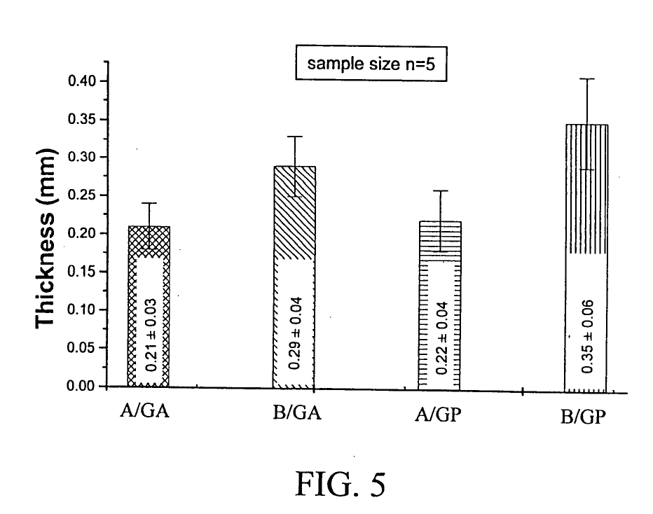

Pericardia tissue chemically treated with glutaraldehyde and genipin shows different characteristics and biocompatibility. FIG. 5 shows thickness of the glutaraldehyde-fixed cellular tissue (A/GA), the glutaraldehyde-fixed acellular tissue (B/GA), the genipin-fixed cellular tissue (A/GP), and the genipin-fixed acellular tissue (B/GP) before implantation. In general, the acellular tissue shows increased tissue thickness by either type of crosslinking (with glutaraldehyde or genipin) as compared to the control cellular tissue. It is further noticed that genipin-fixed acellular tissue shows the highest tissue thickness among the samples characterized, probably due to enhanced water absorption. This high tissue thickness of genipin-fixed acellular tissue is desirable for tissue engineering in vivo or in vitro in medical devices, such as an extended-release drug delivery device, vascular or skin graft, or orthopedic prosthesis of bone, ligament, tendon, and cartilage.

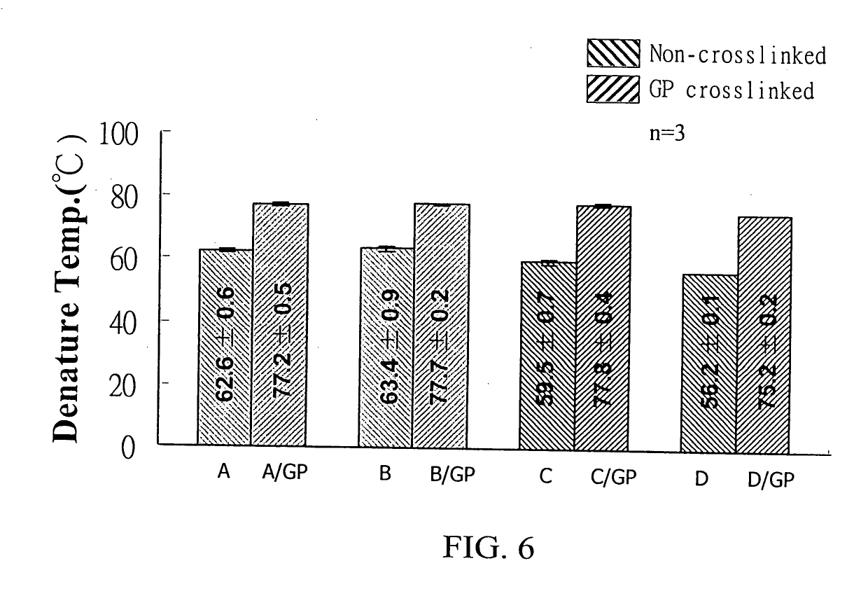

To characterize the degree of tissue crosslinking, denature temperatures are measured on the non-crosslinked and genipin-crosslinked bovine pericardia tissue for (a) specimen-A, cellular tissue; (b) specimen-B, acellular tissue; (c) specimen-C, the acid treated acellular tissue; and (d) specimen-D, the enzyme treated acellular tissue (FIG. 6). The denaturation temperatures of specimens of each studied group before implantation and those retrieved at distinct duration postoperatively are measured in a Perkin Elmer differential scanning calorimeter (Model DSC-7, Norwalk, Conn., USA). This technique was widely used in studying the thermal transitions of collagenous tissues. Details of the methodology used in the denaturation temperature measurement were described elsewhere (J Biomed Mater Res 1998;42:560-567). As shown in FIG. 6, the denature temperatures in all four types of genipin (GP) crosslinked pericardia tissue are higher as expected than their control non-crosslinked counterparts.

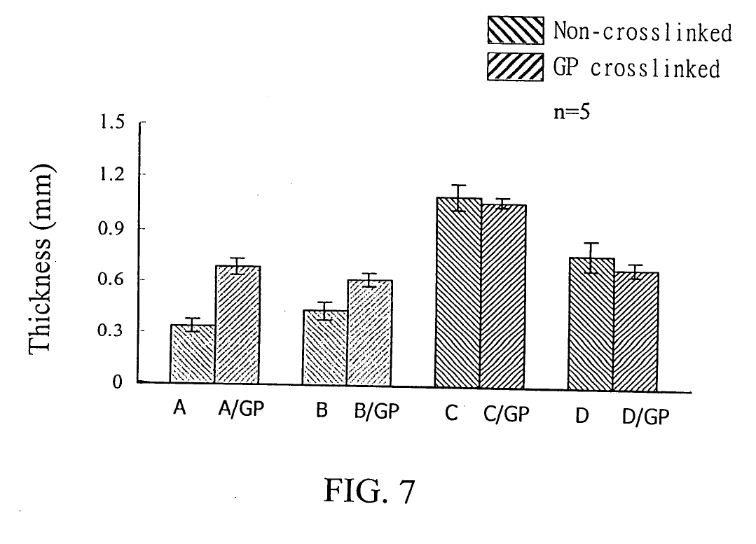

FIG. 7 shows thickness of the bovine pericardia tissue before and after genipin crosslinking for (a) specimen-A, cellular tissue; (b) specimen-B, acellular tissue; (c) specimen-C, the acid treated acellular tissue; and (d) specimen-D, the enzyme treated acellular tissue. For example, a genipin-crosslinked specimen-A is designated as specimen-A/GP, and so forth. It is suggested that thicker tissue is normally due to higher water content or water absorption capability. It implies that the loose extracellular space temporarily occupied by water in acid treated pericardia tissue (in either non-crosslinked tissue or genipin crosslinked tissue) would be desirable for tissue engineering applications in an extended-release drug delivery device, vascular or skin graft, or orthopedic prosthesis, such as bone, ligament, tendon, cartilage, and muscle. The biological tissue material with added porosity may comprise steps of removing cellular material from a natural tissue and crosslinking the natural tissue with a crosslinking agent or with ultraviolet irradiation, wherein the natural tissue is selected from a group consisting of a porcine valve, a bovine jugular vein, a bovine pericardium, an equine pericardium, a porcine pericardium, an ovine pericardium, a valvular leaflet, and submucosal tissue.

### EXAMPLE 4

### Animal Implant Study

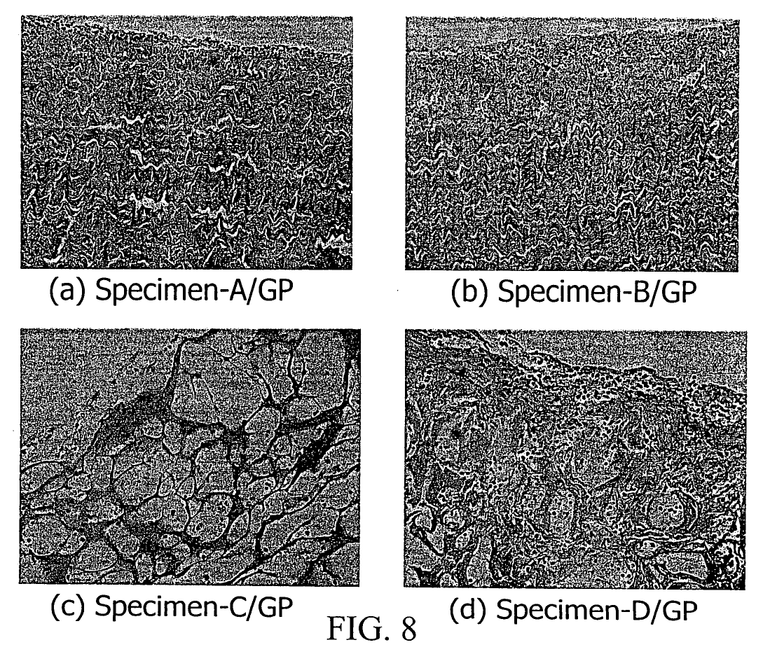

The cellular and acellular tissue fixed with glutaraldehyde and genipin from Example 2 were implanted subcutaneously in a growing rat model (4-week-old male Wistar) under aseptic conditions. Each test sample was approximately 1 cm by 2 cm coupon. In a first study, genipin-crosslinked tissue for specimen-A/GP, specimen-B/GP, specimen-C/GP, and specimen-D/GP are implanted. FIG. 8 shows photomicrographs of H&E stained genipin-crosslinked tissue for (a) specimen-A/GP, cellular tissue; (b) specimen-B/GP, acellular tissue; (c) specimen-C/GP, the acid treated acellular tissue; and (d) specimen-D/GP, the enzyme treated acellular tissue: all retrieved at 3-day postoperatively. It is apparent that cells infiltration into the enlarged pores of the enzyme treated specimen-D/GP is quite visible and evident. The samples used for light microscopy were fixed in 10% phosphate buffered formalin for at least 3 days and prepared for histological examination. In the histological examination, the fixed samples were embedded in paraffin and sectioned into a thickness of 5 μm and then stained with hematoxylin and eosin (H&E). The stained sections of each test sample then are examined using light microscopy (Nikon Microphoto-FXA) for tissue inflammatory reaction and photographed with a 100 ASA Kodachrome film.

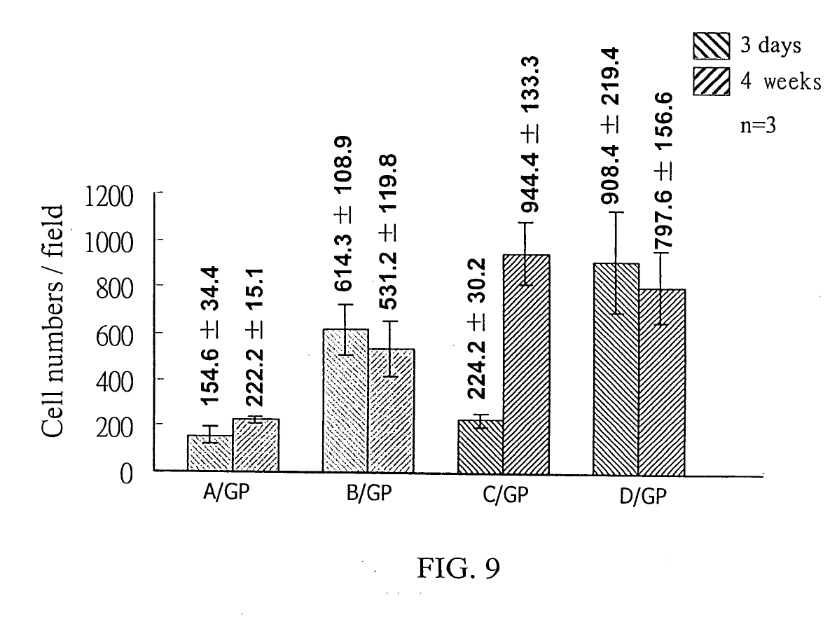

In the first study, genipin-crosslinked tissue for (a) specimen-A/GP, cellular tissue; (b) specimen-B/GP, acellular tissue; (c) specimen-C/GP, the acid treated acellular tissue; and (d) specimen-D/GP, the enzyme treated acellular tissue are retrieved at 3-day and 4 weeks postoperatively. The cell numbers per field (on a reference basis) are counted and shown in FIG. 9. At 4 weeks implantation, both specimen-C/GP and specimen-D/GP show significant higher cells infiltration than the tissue samples without enlarged pores (i.e., specimen-A/GP or specimen-B/GP).

A second study is conducted for comparing the effect of glutaraldehyde (GA)-fixed and genipin (GP)-fixed tissue samples on their ultimate tensile strength. The implanted test samples then were retrieved at 3-day, 1-week, 4-week, 12-week, 24-week, and 52-week postoperatively. At retrieval, the appearance of each retrieved sample first was grossly examined and photographed. The samples were then processed for light microscopy or tensile strength measurement.

The tensile strength values of specimens of each studied group before implantation and those retrieved at distinct implantation duration were determined by uniaxial measurements using an Instron material testing machine (Mini 44, Canton, Mass., USA) at a constant speed of 10 mm/min.

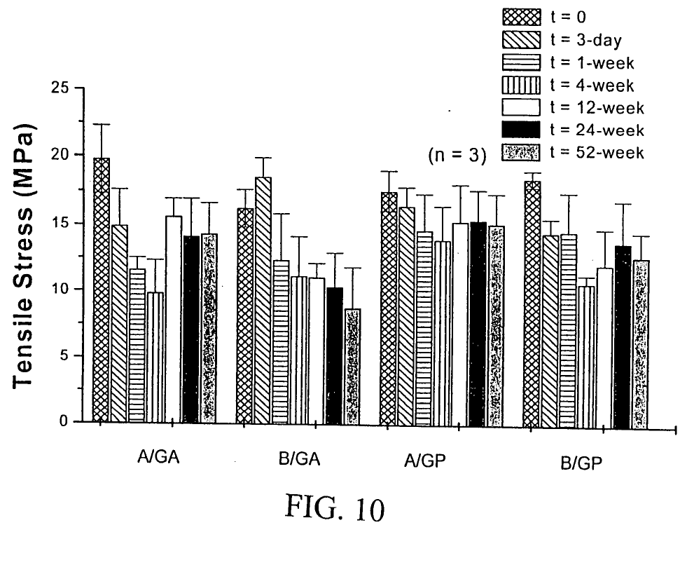

As shown in FIG. 10, the tensile strength values of all test samples before implantation were comparable (P>0.05). It is found that the tensile-strength values of all test samples declined significantly with increasing the duration of implantation prior to 4-week postoperatively (P<0.05). However, with the exception of the glutaraldehyde-fixed acellular tissue, the tensile strength values of all other test samples increased steadily afterwards (P<0.05).

### EXAMPLE 5

### Gelatin Crosslinking Experiment

3-D Scaffold: Gelatin (0.8 g) dissolved in 7 mL phosphate buffered saline was crosslinked by 3 mL 1% genipin or 0.167% glutaraldehyde for 9 hours. The crosslinked gelatin was dried in an oven (37° C.) for 1 hour and then frozen at −30° C. for 9 hours. Finally, the frozen gelatin was lyophilized to create a 3-D scaffold. This represents one type of the “collagen matrix” as defined in the present invention.

In the cell culture study, 16-mm-diameter test samples cut from the sterilized glutaraldehyde-fixed or genipin-fixed tissue were glued to the bottoms of the wells in a 24-well plate (the diameter of each well is about 16 mm) using a sterilized collagen solution. Subsequently, human fibroblasts (HFW) at 5×10⁴ cells/well were seeded evenly on the surface of each test sample in DMEM with 10% FCS. The test samples in the wells then were removed at 3-day through 1-month after cell seeding. During this period, the growth medium was changed routinely. After cell culture, the test scaffolds were washed with phosphate buffered saline (PBS) twice and surviving cell numbers were determined by the MTT assay (J Biomater Sci Polymer Edn 1999;10:63-78).

As disclosed in a co-pending provisional application Ser. No. 60/314,195 filed Aug. 22, 2001 entitled CHEMICAL MODIFICATION OF ACELLULAR BIOMEDICAL MATERIAL WITH GENIPIN, entire contents of which are incorporated herein by reference, the structure of the genipin-fixed scaffold remained intact throughout the entire course of the experiment (up to 1-month after cell culture), while that of the glutaraldehyde-fixed scaffold was found collapsed in the culture medium at 7-day after cell seeding. The human fibroblasts cultured in the genipin-crosslinked scaffold were significantly greater than the glutaraldehyde-crosslinked scaffold throughout the entire course of the experiment as observed in the MTT assay. This indicates that the cellular compatibility of the genipin-crosslinked scaffold is superior to that of the glutaraldehyde-crosslinked scaffold.

The experiment presents the cellular compatibility of a 3-D porous scaffold made from gelatin chemically modified or crosslinked by genipin. The glutaraldehyde-fixed counterpart was used as control. The results obtained indicate that the genipin-crosslinked scaffold had a better cellular compatibility than its glutaraldehyde-fixed counterpart. Additionally, the glutaraldehyde-crosslinked scaffold was found collapsed by 7-day after cell culture, while the genipin-crosslinked scaffold remained intact up to 1-month after cell culture. It is hereby disclosed that the genipin-fixed porous scaffold when configured and adapted for tissue regeneration or tissue engineering comprising steps of removing cellular material from a natural tissue and crosslinking the natural tissue with genipin is desirable, wherein the 3-D scaffold is characterized by reduced antigenicity, reduced immunogenicity and reduced enzymatic degradation upon placement inside a patient's body. The porosity of the scaffold tissue is increased at least 5% over that of the nature tissue adapted for promoting tissue regeneration or tissue engineering

As disclosed and outlined in the co-pending provisional application Ser. No. 60/314,195 by the present inventors, the degrees in inflammatory reaction in the animal studies for the genipin-fixed cellular and acellular tissue were significantly less than their glutaraldehyde-fixed counterparts. Additionally, it was noted that the inflammatory reactions for the glutaraldehyde-fixed cellular and acellular tissue lasted significantly longer than their genipin-fixed counterparts. These findings indicated that the biocompatibility of the genipin-fixed cellular and acellular tissue is superior to the glutaraldehyde-fixed cellular and acellular tissue. It is hypothesized that the lower inflammatory reactions observed for the genipin-fixed cellular and acellular tissue may be due to the lower cytotoxicity of their remaining residues, as compared to the glutaraldehyde-fixed counterparts. In our previous study, it was found that genipin is significantly less cytotoxic than glutaraldehyde (J Biomater Sci Polymer Edn 1999;10:63-78). The cytotoxicity observed for the glutaraldehyde-fixed cellular and acellular tissue seems to result from a slow leaching out of unreacted glutaraldehyde as well as the reversibility of glutaraldehyde-crosslinking. It was observed that when concentrations above 0.05% glutaraldehyde were used to crosslink materials, a persistent foreign-body reaction occurred (J Biomater Sci Polymer Edn 1999;10:63-78).

In the study (co-pending provisional application Ser. No. 60/314,195), it was found that the inflammatory cells were mostly surrounding the cellular tissue, while they were able to infiltrate into the outer layers of the acellular tissue for both the glutaraldehyde-fixed and genipin-fixed groups. As aforementioned, as compared to the cellular tissue, the acellular tissue formed a decreased density of the structural fiber components due to the increase in their thickness (FIG. 5). In addition, after cell extraction, it left more open spaces in the acellular tissue (FIG. 4). As a result, the inflammatory cells were able to infiltrate into the acellular tissue. This significantly increases the contact area between the host immune system (the inflammatory cells) and the foreign material (the acellular-tissue matrix). Consequently, the degrees in inflammatory reaction for the acellular tissue were consistently grater than the cellular tissue.

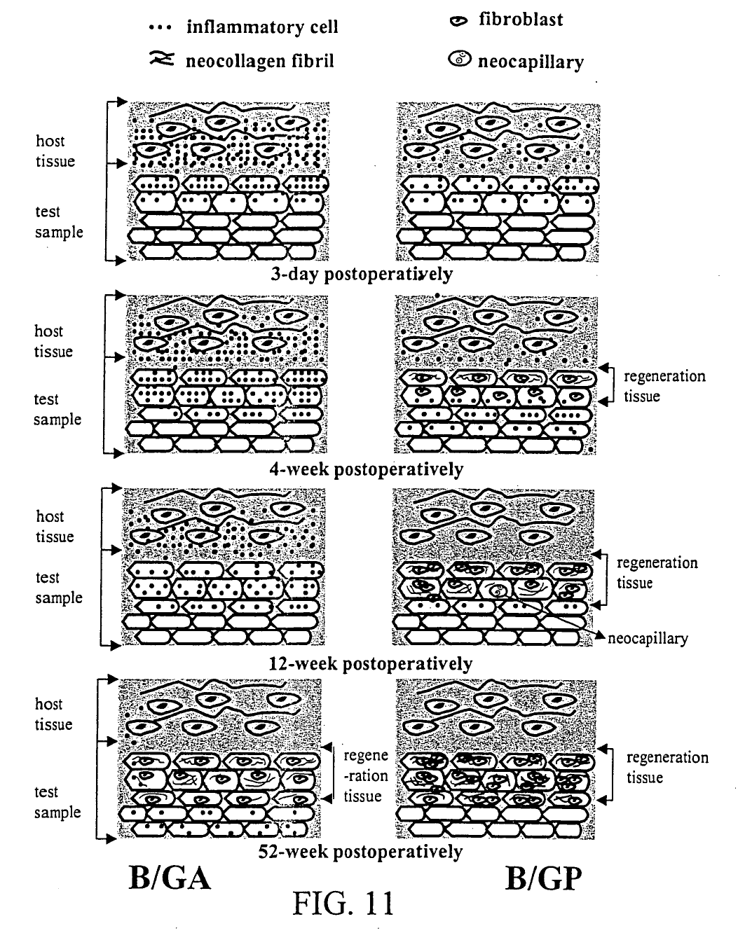

As the cells were able to infiltrate into the outer layers of the acellular tissue, tissue regeneration from the host was observed in this area. FIG. 11 illustrates a suggested mechanism of tissue regeneration in the outer layers of the acellular tissue as per the findings disclosed in the present invention and co-pending provisional application Ser. No. 60/314,195. Once the inflammatory cells infiltrated into the acellular tissue matrix, the enzymes (collagenase and other proteases) secreted by macrophages might start to degrade the fibrous proteins. This allowed fibroblasts from the host tissue (rat's tissue in one example) to migrate into the outer layer of the acellular tissue and to secrete neocollagen fibrils. As duration of implantation progresses, angiogenesis (neocapillaries) occurs. Thus more fibroblasts from the host tissue migrate into the acellular tissue matrix and therefore more neocollagen fibrils are produced. As a result, the most outer layers of the glutaraldehyde-fixed and genipin-fixed acellular tissue observed at 52-week postoperatively were the new tissue regenerated from the host. The tissue regeneration rate observed in the outer layer of the genipin-fixed acellular tissue matrix was significantly faster than its glutaraldehyde-fixed counterpart (FIG. 11).

In conclusion, the results as disclosed in the present invention indicate that the degrees in inflammatory reaction for the genipin-fixed cellular and acellular tissue are significantly less than their glutaraldehyde-fixed counterparts. The acellular tissue provides a natural microenvironment for cell migration to regenerate tissue. The tissue regeneration rate for the genipin-fixed acellular tissue is significantly faster than its glutaraldehyde-fixed counterpart. And this faster tissue regeneration enables a genipin-fixed acellular tissue suitable as a biological scaffold configured and adapted for tissue regeneration or tissue engineering, wherein the scaffold is characterized by reduced antigenicity, reduced immunogenicity and reduced enzymatic degradation upon placement inside a patient's body.

It is hereby disclosed that a method of preparing a biological scaffold configured and adapted for tissue regeneration or tissue engineering comprises steps of removing cellular material from a natural tissue or collagen matrix; and chemically modifying the acellular tissue or collagen matrix with genipin. As defined, “genipin” in this invention is meant to refer to the naturally occurring compound as shown in FIG. 1 and its derivatives, analog, stereoisomers and mixtures thereof. The biological scaffold of the present invention may be characterized by reduced antigenicity, reduced immunogenicity and reduced enzymatic degradation upon placement inside a patient's body. The collagen matrix of the present invention may be selected from a group consisting of an insoluble collagen, a soluble collagen, an atelocollagen prepared by removing telopeptides on the collagen molecule terminus using protease other than collagenase, a chemically modified collagen obtained by succinylation or esterification of above-described collagens, a collagen derivative such as gelatin, a polypeptide obtained by hydrolysis of collagen, and a natural collagen present in natural tissue (ureter, blood vessel, pericardium, heart valve, etc.).

It is further disclosed that a biological scaffold for cells seeding, cell growth or cell proliferation may comprise a natural tissue devoid of cellular material and chemically modified by genipin. As indicated in FIG. 4, the porosity increase of the acellular specimen-B is 7.6% higher than its control cellular specimen-A. Furthermore, the porosity increase of the acid treated acellular tissue specimen-C and the porosity increase of the enzyme treated acellular tissue specimen-D are 53% and 61%, respectively higher than the porosity of the control cellular specimen-A. The biological scaffold may be characterized by an increase of the biological scaffold volume after treatment by at least 5%, preferably more than 10% of volume porosity change (FIG. 4). The “treatment” to make a biological tissue material or scaffold of the present invention may include the acellularization process, acid treatment, base treatment, and/or enzyme (e.g. protease) treatment processes. The biological tissue material is selected from a group consisting of a tissue valve, a tissue valve leaflet, a vascular graft, a ureter, a urinary bladder, pericardium, and a dermal graft.

It is another embodiment of the present invention to provide a tendon or ligament graft for use as connective tissue substitute, the graft being formed from a segment of connective tissue protein, wherein the segment is crosslinked with genipin, its analog or derivatives. The connective tissue protein may be collagen or pericardia patches that is substantially devoid of cells and porosity of the tissue graft is increased at least 5% adapted for promoting autogenous ingrowth into the graft. The process for using a tissue sheet to make a tendon or ligament graft has been disclosed by Badylak et al. in U.S. Pat. No. 5,573,784, No. 5,445,833, No. 5,372,821, No. 5,281,422, and so forth, the entire contents of which are incorporated herein by reference, which disclose a method for promoting the healing and/or regrowth of diseased or damaged tissue structures by surgically repairing such structures with a tissue graft construct prepared from a segment of intestinal submucosal tissue.

Further, Badylak et al. in U.S. Pat. No. 6,485,723, the entire contents of which are incorporated herein by reference, discloses an improved tissue graft construct comprising vertebrate submucosa delaminated from both the external smooth muscle layers and the luminal portions of the tunica mucosa and added primary cells, wherein the vertebrate submucosa comprises tunica submucosa delaminated from both the tunica muscularis and at least the luminal portion of the tunica mucosa of vertebrate intestinal tissue. With added porosity, it is herein provided a biological tissue material derived from submucosal tissue adapted for promoting tissue regeneration.

U.S. Pat. No. 6,506,398 issued to Tu (a co-inventor of the present invention), the entire contents of which are incorporated herein by reference, discloses a vascular graft comprising Vascular Endothelial Growth Factor (VEGF) and/or Platelet Derived Growth Factor (PDGF) for enhanced site-specific angiogenesis and methods thereof. At least one VEGF, PDGF or angiogenesis factor is incorporated into the vascular graft to facilitate enhanced angiogenesis so as the cells are stimulated to migrate to environments having higher concentration of growth factors and start mitosis. With added porosity, it is provided a biological tissue material with loaded growth factors adapted for promoting tissue regeneration, wherein the growth factor is selected from the group consisting of VEGF, VEGF 2, bFGF, VEGF121, VEGF165, VEGF189, VEGF206, PDGF, PDAF, TGF-β, PDEGF, PDWHF, and combination thereof.

Vascular endothelial growth factor (VEGF) is mitogenic for vascular endothelial cells and consequently is useful in promoting neovascularization (angiogenesis) and reendothelialization. Angiogenesis means the growth of new capillary blood vessels. Angiogenesis is a multi-step process involving capillary endothelial cell proliferation, migration and tissue penetration. VEGF is a growth factor having a cell-specific mitogenic activity. It would be desirable to employ a wound healing substrate incorporating a mitogenic factor having mitogenic activity that is highly specific for vascular endothelial cells following vascular graft surgery, balloon angioplasty or to promote collateral circulation. U.S. Pat. No. 5,194,596 discloses a method for producing VEGF while U.S. Pat. No. 6,040,157 discloses a specific VEGF-2 polypeptide. Both patents are incorporated herein by reference.

Gordinier et al. in U.S. Pat. No. 5,599,558 discloses a method of making a platelet releasate product and methods of treating tissues with the platelet releasate. Platelet derived growth factor (PDGF) is a well-characterized dimeric glycoprotein with mitogenic and chemoattractant activity for fibroblasts, smooth muscle cells and glial cells. In the presence of PDGF, fibroblasts move into the area of tissue needing repair and are stimulated to divide in the lesion space itself. It has been reported that the cells exposed to lower PDGF concentrations are stimulated to move to environments having higher concentrations of PDGF and divide. The patent is incorporated hereby by reference.

In some aspects, there is provided a method for promoting autogenous ingrowth of a biological tissue material comprising the steps of providing a natural tissue, removing cellular material from the natural tissue, increasing porosity of the natural tissue by at least 5%, loading an angiogenesis agent or autologous cells into the porosity, and crosslinking the natural tissue with a crosslinking agent. In one preferred embodiment, the angiogenesis agent is ginsenoside Rg₁, ginsenoside Re or selected from the group consisting of VEGF, VEGF 2, bFGF, VEGF121, VEGF165, VEGF189, VEGF206, PDGF, PDAF, TGF-β, PDEGF, PDWHF, and combination thereof.

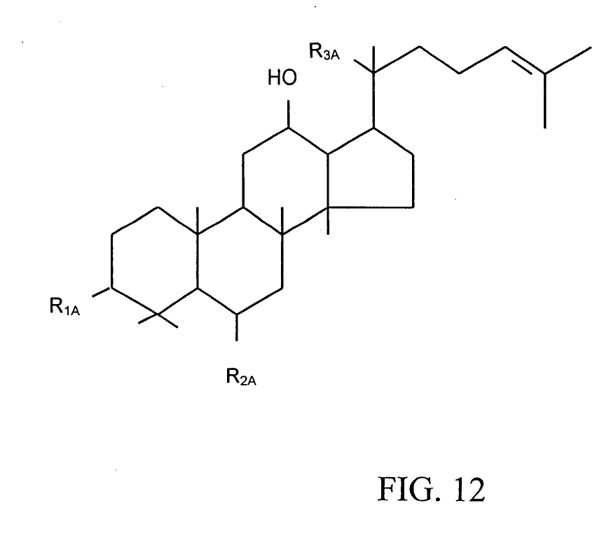

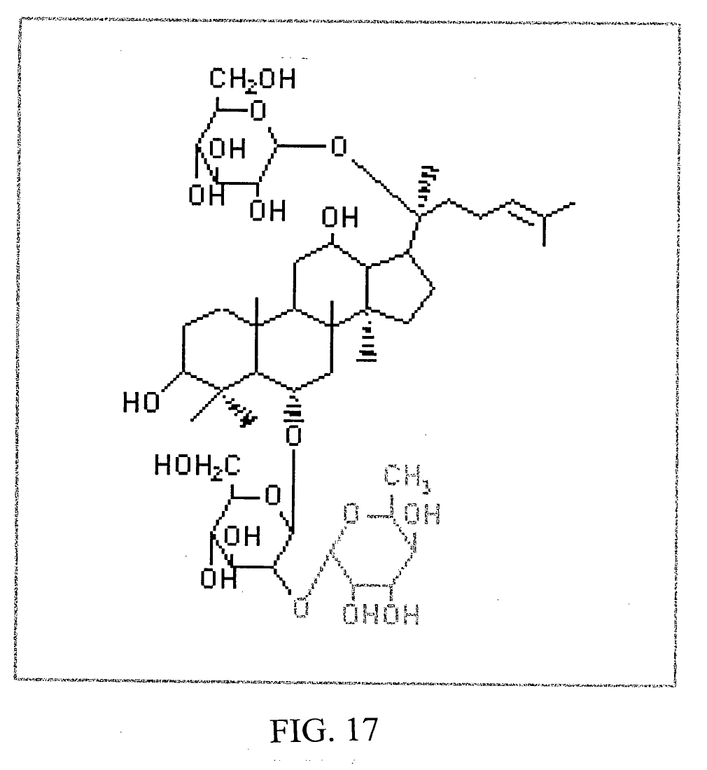

It is known that protein type growth factors have relatively short shelf life. For medical device use, it is one object of the invention to provide an organic compound, non-protein type growth factors, such as ginsenoside Rg₁ (as shown in FIG. 12) and/or ginsenoside Re (as shown in FIG. 17).

Ginseng is one of the most widely used herbal drugs and is reported to have a wide range of therapeutic and pharmacological activities. The two major species of commerce are Panax ginseng C. A. Meyer (Asian ginseng), and Panax quinquefolius L. (North American ginseng). Both species contain active ginsenoside saponins, but there are significant differences in their identity and distribution. It has been observed that over thirty ginsenosides have been identified from Panax spp., however six of these, Rg₁, Re, Rb₁, Rc, Rb₂, and Rd constitute the major ginsenosides accounting for over 90% of the saponin content of ginseng root. Standard ginsenosides Rg₁, Re, Rb₁, Rc, Rb₂ and Rd can be isolated and characterized by NMR. In contrary to general angiogenesis effects of ginsenoside Rg₁ and Re, ginsenoside Rg₃ can block angiogenesis and inhibit tumor growth and metastasis by downregulating the expression of VEGF mRNA and protein and reducing microvascular density. Some aspects of the invention relate to a method of reducing angiogenesis for treating tissue comprising: providing crosslinkable biological solution to the target tissue, wherein the crosslinkable biological solution is loaded with at least one anti-angiogenic agent (also known as angiogenic antagonist or inhibitor) such as ginsenoside Rg₃ and the like.

Duckett et al. in U.S. Pat. No. 6,340,480, the entire contents of which are incorporated herein by reference, discloses a composition for promoting circulation, comprising an effective amount of L-arginine, ginseng and Ziyphi fructus, the constituents being administered to stimulate release of NO in the body. Some past studies with natural ingredients have shown that with natural medicines include ginseng, ginsenoside, and its purified derivative Rg₁ (also known as RG-1) have a tendency to increase synthesis of NO levels. It has been shown that Rg₁ enhances the production of NO for killing certain tumor cells. See, e.g., Fan et al., Enhancement of Nitric Oxide Production from Activated Macrophages by a Purified Form of Ginsenoside (Rg₁), American Journal of Chinese Medicine, Vol. XXHI, Nos. 3-4. pp. 279-287 (1995 Institute for Advanced Research in Asian Science and Medicine).

FIG. 12 shows a chemical formula of ginsenoside Rg₁, one of the principal active components of ginseng saponins which is isolated from the roots of Panax ginseng. In one embodiment as shown in FIG. 12, in which R_{1A}═OH or O-Glc, R_{2A}═H or O-Glc, R_{3A}═O-Glc, wherein Glc designates a β-D glucopyranosyl group. Rg₁ is believed to stimulate vascular endothelial cells proliferation, and tube formation in a patient. Ginseng's therapeutic uses were recorded in the oldest Chinese pharmacopeia, Shen Nong Ben Cao Jing, written about two thousand years ago. Ginseng action is non-local and non-specific. In Asian medicine, ginseng is used as a tonic to revitalize the function of organism as a whole and replenish vital energy (“chi”). It is traditionally used as the best supplemental and restorative nature agent during convalescence and as a prophylactic to build resistance, reduces susceptibility to illness, and promotes health and longevity.

Other functions of ginseng are to stimulate mental and physical activity, strengthen and protect human organism, increase physical and mental efficiency and to prevent fatigue. Ginseng has good effect on the stomach, the brain, and the nervous system. Ginseng is effective for reflex nervous disease. Ginseng has also been found to have an anti-cancer effect. There are more than 30 kinds of ginsenosides, and each one function differently. Ginsenoside Rh₂ has anti-tumor activity. Ginsenoside Rg₁ can enhance DNA and RNA formation, which may speed up the angiogenesis. In some aspect of the present invention, there is provided a method for promoting autogenous ingrowth of a biological tissue material comprising the steps of providing a natural tissue, removing cellular material from the natural tissue, increasing porosity of the natural tissue by at least 5%, loading an angiogenesis agent or autologous cells into the porosity, and crosslinking the natural tissue with a crosslinking agent. In one preferred embodiment, the angiogenesis agent is ginsenoside Rg₁. In still another aspect of the invention, there is provided a method for treating cancer or tumor by implanting a biological tissue material comprising the steps of providing a natural tissue, removing cellular material from the natural tissue, increasing porosity of the natural tissue by at least 5%, loading a cancer/tumor antagonist agent into the porosity, and crosslinking the natural tissue with a crosslinking agent. In one preferred embodiment, the cancer/tumor antagonist is ginsenoside Rh₂.

Some aspects of the invention relate to a method for promoting angiogenesis for treating tissue comprising: providing crosslinkable biological solution to the target tissue, wherein the crosslinkable biological solution is loaded with at least one angiogenic agent (also known as angiogenic growth factor) such as ginsenoside Rg₁. Some aspects of the invention relate to a method for treating cancer or tumor of a patient comprising: providing crosslinkable biological solution to the target tissue, wherein the crosslinkable biological solution is loaded with at least one cancer/tumor antagonist agent such as ginsenoside Rh₂.

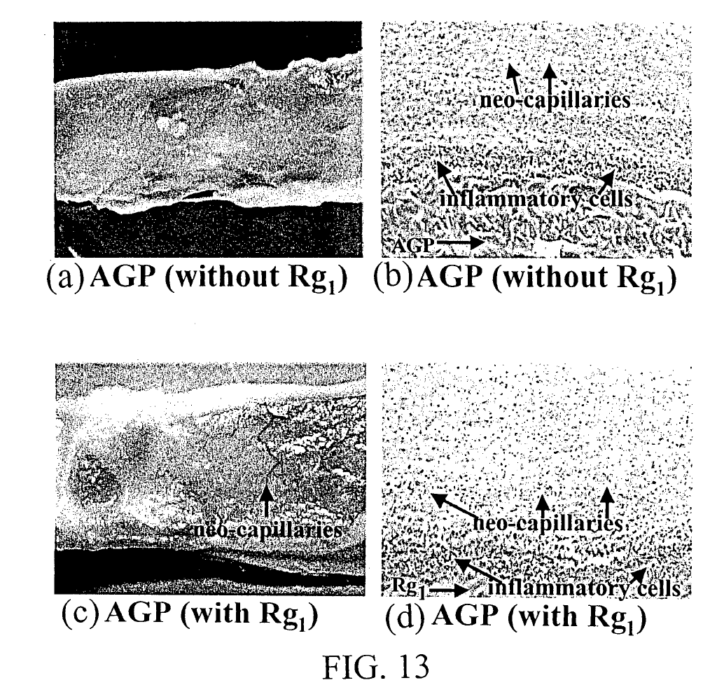

FIG. 13 show cells infiltration extents of genipin-crosslinked acellular bovine pericardia tissue with angiogenesis factors for (a) specimen-AGP, without Rg₁; (b) light microscopy of specimen a (specimen-AGP, without Rg₁); (c) specimen-AGP, with Rg₁; and (d) light microscopy of specimen c (specimen-AGP, with Rg₁); wherein all implants are retrieved at 1-week postoperatively. The micro-vessel numbers per field (on a reference basis) are measured under a microscope using an imaging processing software. The micro-vessel density for the Rg₁ loaded explant (specimen (b) in FIG. 13) is 778 vessels/mm² that is statistically significantly higher than the micro-vessel density for the control explant (specimen (d) in FIG. 13) of 341 vessels/mm².

In some aspects of the present invention, the acellular tissue structure with a porosity increase of more than 5% is also suitable for use in anti-adhesion patches for abdominal surgery, anti-adhesion patches for cardiovascular surgery, acellular matrix for regeneration of myocardiocytes, and vascular grafts. Rg₁ has shown properties of stimulating HUVEC proliferation, tube formation and chemoinvasion in in vitro studies (T.P.Fan at 3^{rd} Asian International Symposium on Biomaterials and Drug Delivery Systems, Apr. 16, 2002). Some aspects of the invention relate to a method for promoting angiogenesis comprising loading ginsenoside Rg₁ and/or ginsenoside Re onto an acellular tissue or loading ginsenoside Rg₁ and/or ginsenoside Re onto a wound dressing device in wound care.

Myocardial Tissue Regeneration

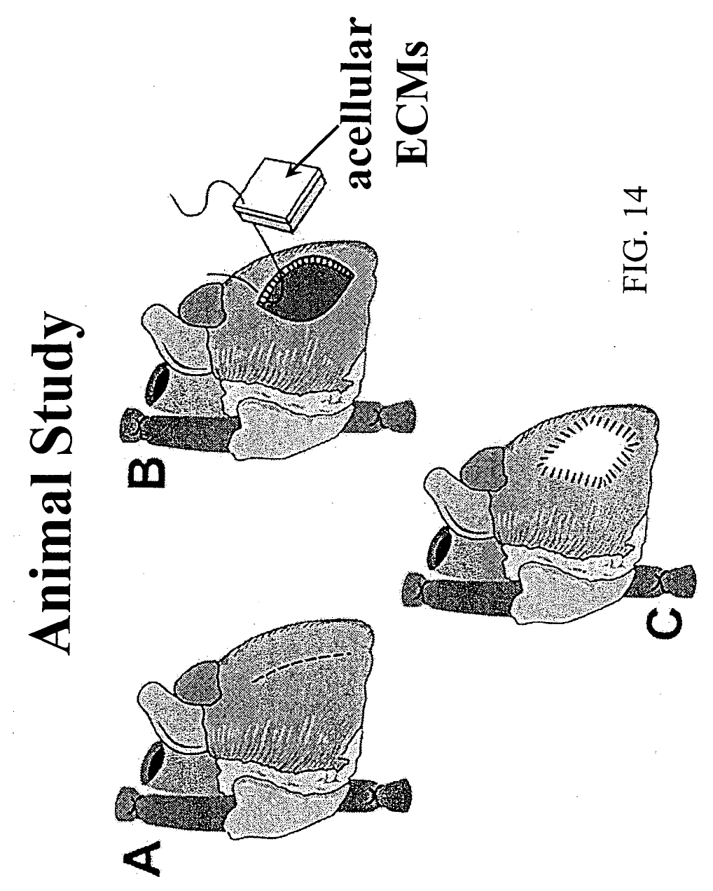

The current material for myocardial artery includes Dacron polyester fabric, expanded polytetrafluoroethylene (e-PTFE), glutaraldehyde-treated bovine pericardium, anti-biotic preserved or cryopreserved homografts. Material related failures include no cell growth, not viable, no pulsatile flow, being treated as a foreign body, thrombogenic nature, and infectable. The animal model (shown in FIG. 14) is a transmural defect surgically created in the right ventricle of an adult rat. The test specimen is an acellular tissue patch fixed with genipin at about 60% crosslinkage and the control is e-PTFE patch. Each specimen is 0.7 cm in width and 0.7 cm in height. The implant specimens are retrieved postoperatively at 4 weeks (sample size=5) and one month.

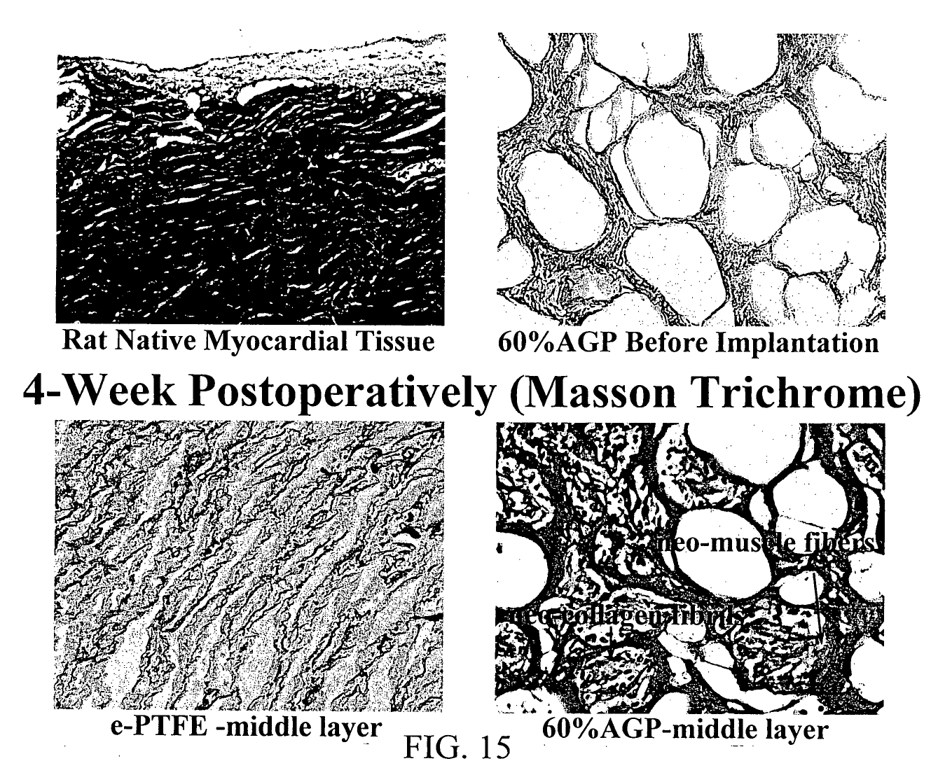

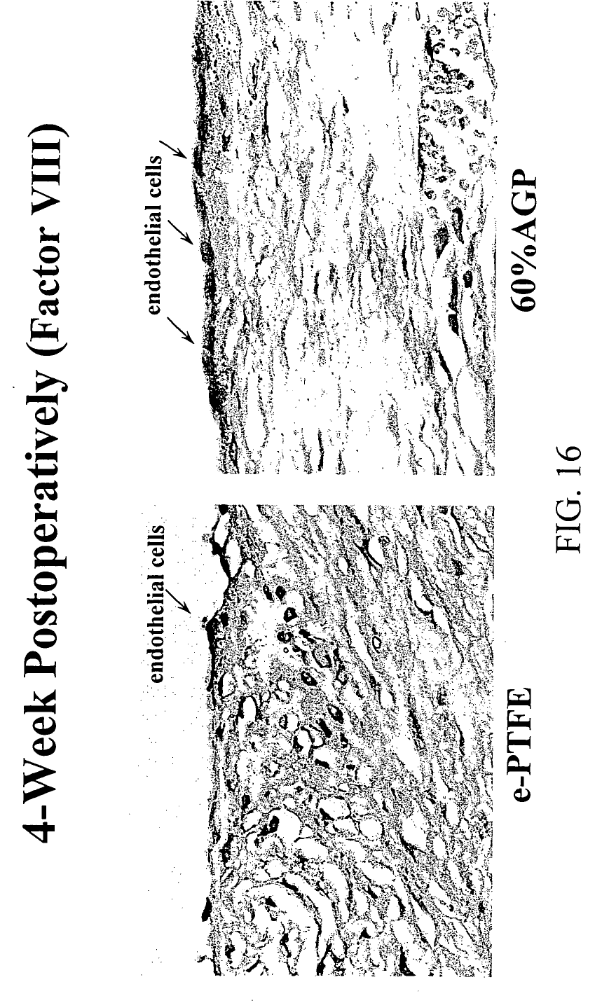

FIG. 15 shows 4-week postoperative results on animal myocardial patch study of FIG. 14: photomicrographs of Masson Trichrome stained explant while FIG. 16 shows photomicrographs of Factor VIII stained explant. The middle layer of the 60% crosslinkage acellular tissue patch fixed with genipin is abundantly filled with neo-muscle fibers and neo-collagen fibrils as evidenced by Masson Trichrome stain. The blood-contacting tissue surface for the 60% crosslinkage acellular tissue patch fixed with genipin is filled with contagious endothelial cells while the control e-PTFE implant is with sparse endothelialization. It is concluded that acellular biological tissue fixed with genipin is a promising tissue-engineering extracellular matrix for repairing myocardial defect.

### EXAMPLE 6

### In vivo Angiogenesis study with Ginsenoside

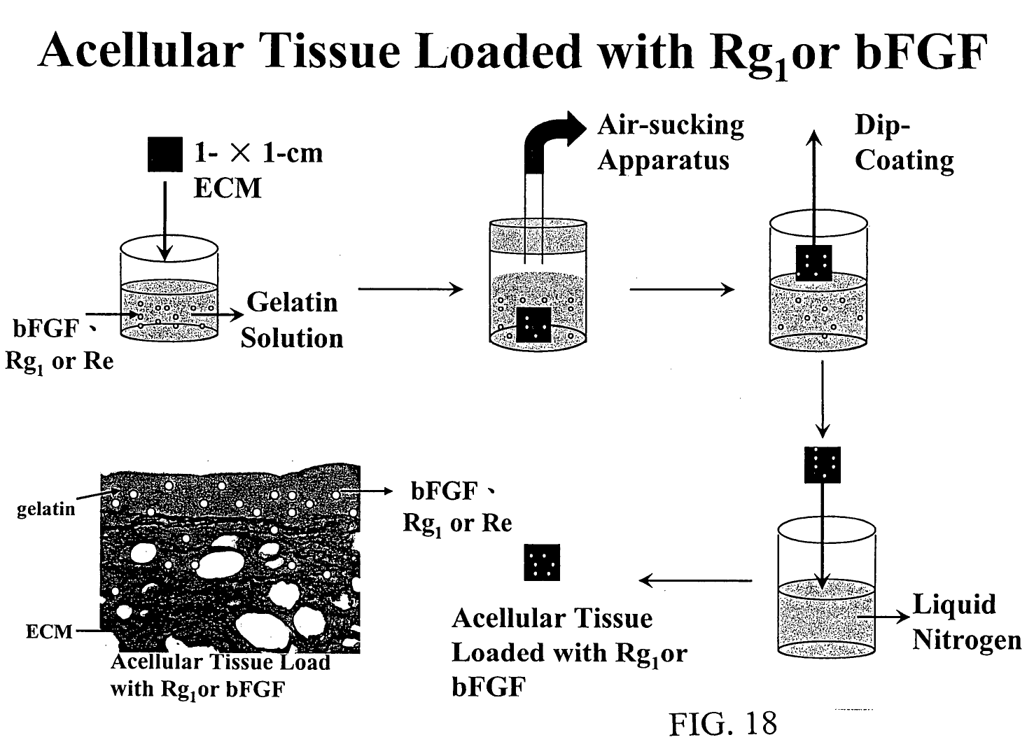

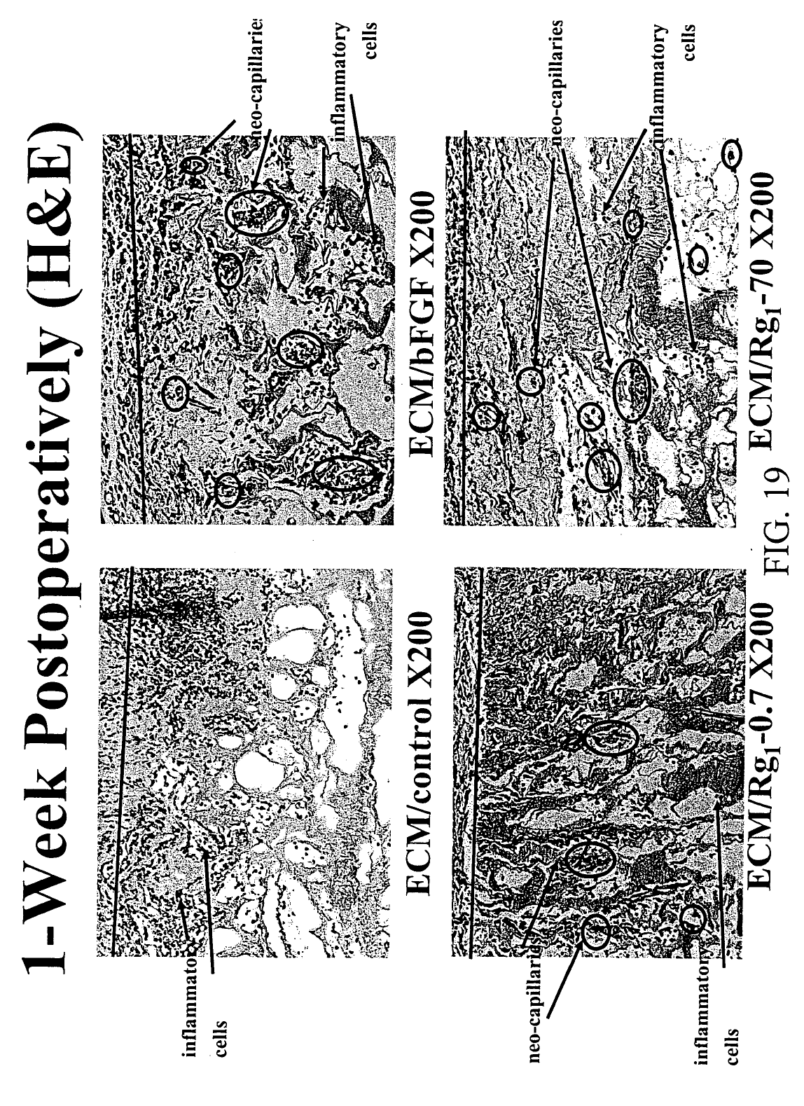

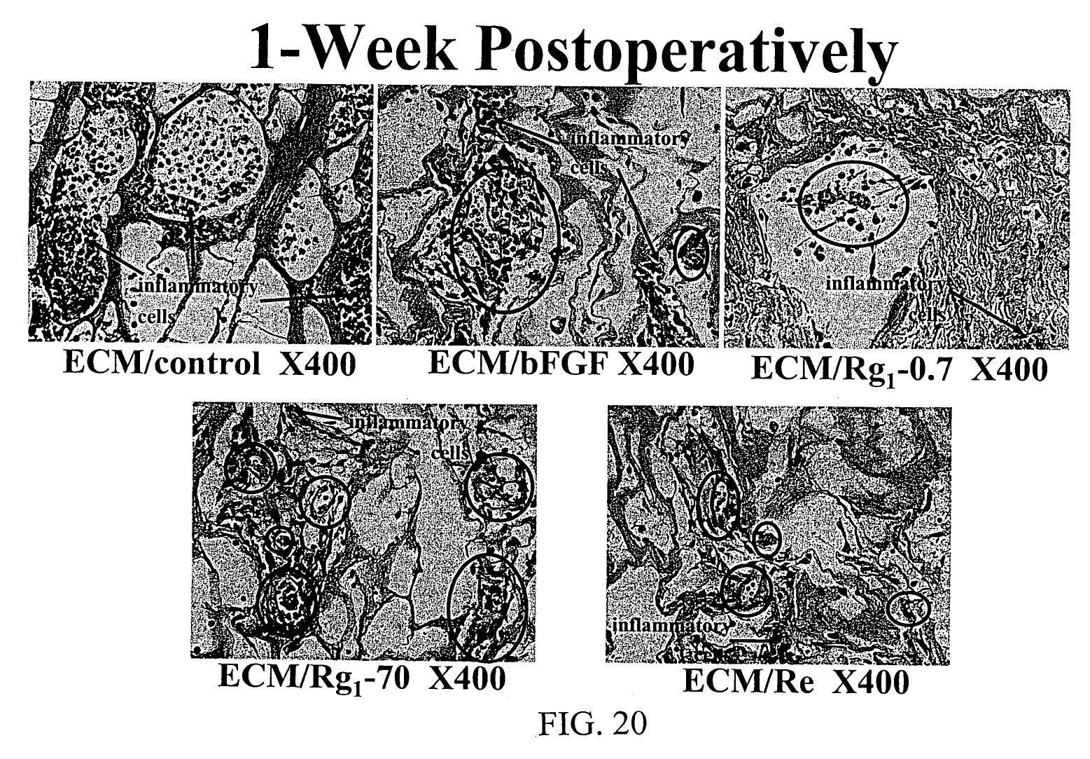

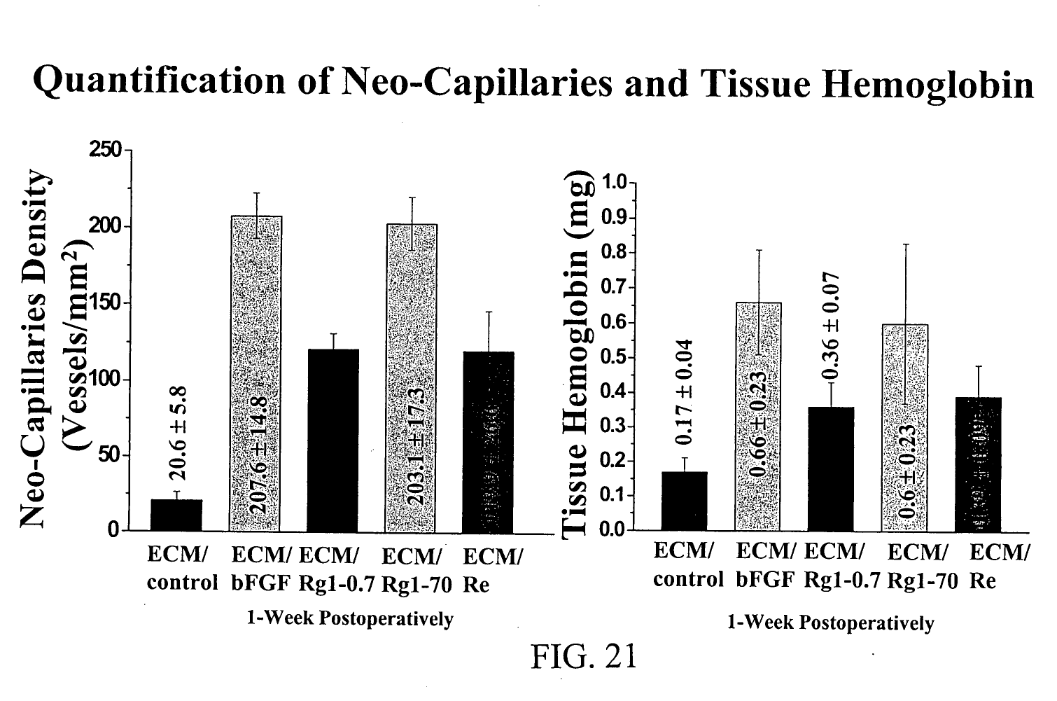

The primary challenge for tissue engineering vital organs is the requirement for a vascular supply for nutrients and metabolite transfer. FIG. 18 shows a preparation method of loading an acellular tissue with ginsenoside Rg₁ or ginsenoside Re (both are organic compound growth factors), or bFGF (a protein type growth factor which has a short shelf life). As shown in FIG. 18, extracellular membranes of 1-cm by 1-cm specimens are used to load model growth factors onto the specimens by air-sucking, dip coating and liquid nitrogen cooling steps. The animal implant study includes a rat intramuscular model, wherein the test groups are loaded with 0.7 μg Rg₁, 0.7 μg bFGF, 70 μg Rg₁ or 70 μg Re growth factors. FIG. 19 shows 1-week postoperative results on animal angiogenesis study: photomicrographs of H&E (hematoxylin and eosin) stained tissue explant while FIG. 20 shows photomicrographs of SEM tissue explant. FIG. 21 shows 1-week postoperative results on animal angiogenesis study: quantification of neo-capillaries and tissue hemoglobin. Both organic compound growth factor and protein growth factor promote angiogenesis as evidenced by enhanced neo-capillaries and tissue hemoglobin measurements as compared to control. However, the protein growth factors tend to have a shorter shelf life than the organic growth factors.

Biological Solution Kits

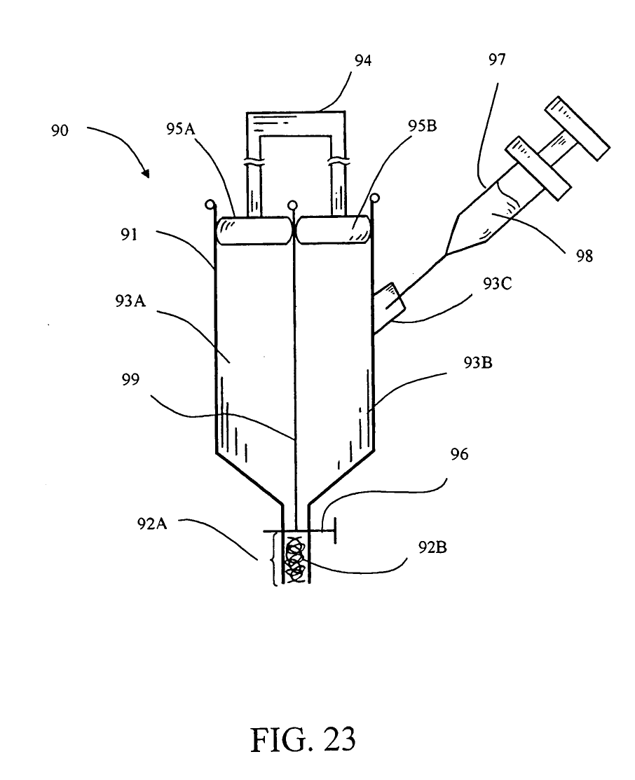

FIG. 23 shows a crosslinkable biological solution kit 90 comprising a first crosslinkable biological solution component 93B and a second crosslinker component 93A. The kit has a double-barrel cylinder 91 with a divider 99 that separates the crosslinkable biological solution component 93B from the crosslinker component 93A before use, wherein each barrel is appropriately sized and configured to provide a desired amount and ratio of each component for later mixing and application. The kit further comprises an end portion 92A with (optionally) appropriate mixing means 92B for mixing the liquid/solution from each of the double-barrel. A control valve 96 is provided to maintain the components 93A, 93B in their own barrels before use or is activated to start the mixing process. The plunger means 94 for pressurizing the components 93A, 93B toward the end portion 92A has a first plunger 95A and a second plunger 95B. In an alternate embodiment, the plunger means 94 can be either mechanical or equipped with a gas or liquid compressor. In one preferred embodiment, the mixed solution can be sprayed onto an implant or a stent. In another embodiment, the mixed solution is used directly onto a target tissue. In a further embodiment, the cylinder comprises a liquid input port 93C, wherein the bioactive agent(s) 98 can be injected via the injecting applicator 97 into and mixed with the crosslinkable biological solution component 93B.

### EXAMPLE 7

### Biological Solution as Medical Material

The first step for preparing a biological solution as medical material is to load the double-barrel cylinder with 4 mg/ml collagen solution at a pH4 as crosslinkable biological solution component 93B. The second step is to load 0.5% genipin solution as the crosslinker component 93A. Each of the double-barrel is appropriately sized and configured to provide a desired ratio and amount of each component 93A, 93B for later mixing in the end portion 92A. One example is to provide 0.6 ml of component 93A with respect to 4 ml of component 93B. Upon receiving the cylinder in sterile conditions, an operator as end-users prepares a paclitaxel solution (Solution A) by mixing 20 mg paclitaxel in one ml absolute alcohol, wherein Solution A is readily mixed into the component 93B by the operator. Paclitaxel is used as a bioactive agent in this example. When use, two barrels are pushed to mix the component 93A and component 93B that contains the desired bioactive agent. In one embodiment, the mixed crosslinkable biological solution is loaded onto a stent at about 30° C. temperature and subsequently leave the coated stent at 37° C. to solidify collagen, evaporate acetic acid, and crosslink collagen on the stent. The loading process may comprise spray coating, dip coating, plasma coating, painting or other known techniques. In another embodiment, the crosslinkable biological solution is administered or delivered to the target tissue accompanied with means for adjusting the biological solution to pH7, either by removing excess acetic acid or by neutralizing with a base solution.

Peritoneal Regeneration

Clinically, the incidence of intra-abdominal adhesions ranges from 67% to 93% after general surgical abdominal operations and up to 97% after open gynecologic pelvic procedures (Becker J M, et al., J Am Coll Surg 1996;183:297). Adverse sequelae associated with postsurgical abdominal adhesions include bowel obstruction, difficult reoperative surgery, chronic pain, and infertility in women. Placing surgical-repair materials as a physical barrier between the injured peritoneum and its adjacent organs is a direct approach to prevent intra-abdominal adhesions. Various surgical-repair materials made of natural or synthetic polymers have been reported to be effective in reducing postoperative abdominal adhesions (Matsuda S et al., Biomaterials 2002;23:2901).

However, design of optimal surgical-repair materials to reinforce or replace peritoneal tissues remains problematic. The conventional knitted polypropylene mesh is known to suffer from a number of complications. Degradable natural or synthetic polymers such as Seprafilm™ (Genzyme, Cambridge, Mass.) or collagenous films have been used as surgical-repair materials (Abraham G A et al., J Biomed Mater Res 2000;51:442). However, these degradable prostheses cannot provide sufficient mechanical strength during the degradation process. Additionally, the effectiveness of degradable barriers to reduce adhesion formation is questionable. It was reported that an ideal prosthesis as a surgical-repair barrier should be able to maintain its strength, integrate with surrounding tissue, and not induce adhesion formation (Bellon J M et al., J Surg 2001;25:147). Unfortunately, prior art does not teach or provide such a biomaterial.

U.S. Pat. No. 6,545,042, entire contents of which are incorporated herein by reference, discloses bovine pericardia as a biomaterial to manufacture various bioprostheses because of their inherent strength and biocompatibility. In the study, a cell extraction process was employed to remove the cellular components from bovine pericardia. It was reported that tissue extraction may decrease its antigenic load when implanted in vivo (Courtman D W et al., J Biomed Mater Res 1994;28:655). The acellular bovine pericardia were fixed with a naturally occurring crosslinking agent, genipin, as a novel surgical-repair material. It was found in our previous study that the cytotoxicity of genipin is significantly lower than glutaraldehyde (Sung et al., J Biomater Sci Polymer Edn 1999;10:63). Additionally, it was demonstrated that the genipin-fixed tissues have a significantly better biocompatibility than their glutaraldehyde-fixed counterparts in several animal studies (Chang Y et al., Biomaterials 2002;23:2447).

In the following examples from a study, the feasibility of using acellular bovine pericardia fixed with genipin or glutaraldehyde as a surgical-repair material to fix an abdominal wall defect created in a rat model was evaluated. The genipin-fixed cellular counterpart and a commercially available polypropylene mesh (Marlex®) and a hyaluronate/carboxymethylcellulose-complex membrane (Seprafilm™) were used as controls. The implanted samples were retrieved at 3-day, 1-month, and 3-month postoperatively. The degrees of abdominal adhesion, calcification, inflammatory reaction, and tissue regeneration of each retrieved sample were evaluated and compared.

### EXAMPLE 8

### Test Samples for Peritoneal Regeneration Study

Bovine pericardia procured from a slaughterhouse were used as raw materials. The procedure used to remove the cellular components from bovine pericardia was based on a method developed by Courtman et al. with slight modifications (Courtman D W et al., J Biomed Mater Res 1994;28:655) and is disclosed in U.S. Pat. No. 6,545,042. Bovine pericardia first were immersed in a hypotonic tris buffer (pH 8.0) containing a protease inhibitor (phenylmethyl-sulfonyl fluoride, 0.35 mg/L) for 24 hours at 4° C. with constant stirring. Subsequently, they were immersed in a 1% solution of Triton X-100 (octylphenoxypolyethoxyethanol, Sigma Chemical Co., St. Louis, Mo.) in tris-buffered salt solution with protease inhibition for 24 hours at 4° C. with constant stirring. Samples then were thoroughly rinsed in Hank's physiological solution and digested with DNase and RNase at 37° C. for 1 hour. This was followed by a further 24 hours extraction with Triton-X 100 in tris buffer. Finally, all samples were washed for 48 hours in Hanks' solution.

Cellular and acellular tissues were fixed in a 0.625% aqueous glutaraldehyde (Merck KGaA, Darmstadt, Germany) solution or a 0.625% aqueous genipin (Challenge Bioproducts, Taichung, Taiwan) solution at 37° C. for 3 days. The aqueous glutaraldehyde and genipin solutions were buffered with phosphate buffered saline (PBS, 0.1M, pH 7.4, Sigma Chemical Co.). The degree of crosslinking for each studied group was determined by measuring its fixation index and denaturation temperature (n=5). The fixation index, determined by the ninhydrin assay, was defined as the percentage of free amino groups in test tissues reacted with glutaraldehyde or genipin subsequent to fixation. The denaturation temperature of each studied group was measured by a Perkin-Elmer differential scanning calorimeter (model DSC-7, Norwalk, Conn., USA). Details of the methods used in the determinations of fixation index and denaturation temperature of test tissues were previously described (Sung H W et al., J Biomed Mater Res 1999;47:116).

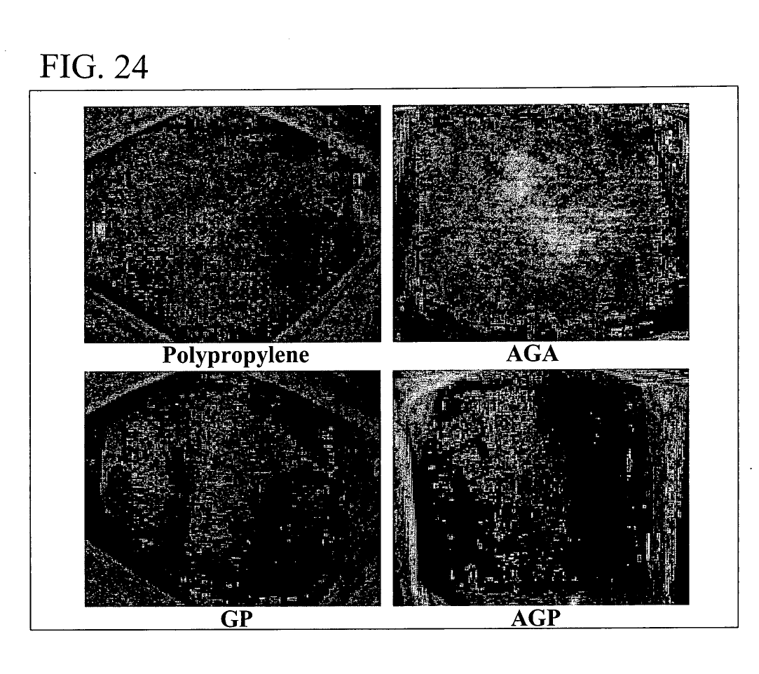

FIG. 24 show photographs of the implanted polypropylene mesh and the AGA, GP, and AGP patches. After fixation, it was found that the color of the glutaraldehyde-fixed tissue (AGA) turned yellowish, while the genipin-fixed tissues (GP and AGP) became dark-bluish. The fixation indices (and denature temperatures) of the AGA, GP, and AGP patches were 92.2±0.7% (85.1±0.3° C.), 91.5±1.0% (77.2±0.5° C.), (77.8±0.2° C.), respectively. The fracture tension values for the AGA (6.8±0.7 kN/m), GP (6.4±0.5 kN/m), and AGP (6.3±0.8 kN/m) patches were approximately the same (p>0.05).

### EXAMPLE 9

### Animal Study for Peritoneal Regeneration Study

The test samples evaluated in the animal study were: the glutaraldehyde-fixed acellular tissue (AGA), the genipin-fixed acellular tissue (AGP), and the genipin-fixed cellular tissue (GP). Test samples were sterilized in a graded series of ethanol solutions with a gradual increase in concentration from 20% to 75% over a period of 4 hours. Subsequently, they were thoroughly rinsed in sterilized PBS for approximately 1 day, with a solution change several times. A knitted polypropylene mesh (Marlex®, Ethicon, Sommeville, N.J., USA) and a Seprafilm™ membrane (Genzyme, Cambridge, Mass., USA) were used as controls. Seprafilm™ is a hydrophilic membrane composed of sodium hyaluronate and carboxymethylcellulose.

The animal study was conducted under aseptic conditions using a growing rat model (4-week-old male Wistar). Rats were anesthetized by intramuscular injection of sodium pentobarbital (30 mg/kg). Defects (4×4 cm²) involving all the layers of the abdominal wall including the parietal peritoneum (with the exception of the skin and subcutaneous soft tissue) were created in the abdominal wall of anesthetized rats. Subsequently, the created defects were repaired by each studied group of a similar size using a 4-0 silk suture (FIG. 24). Skin closure was finally obtained with 3-0 silk continuous sutures. For the test groups (AGA, GP, and AGP), the implanted samples were retrieved at 3-day, 1-month, and 3-month (n=5) postoperatively. For the control groups (polypropylene mesh and Seprafilm™), the implanted samples were retrieved at 3-month postoperatively (n=5).

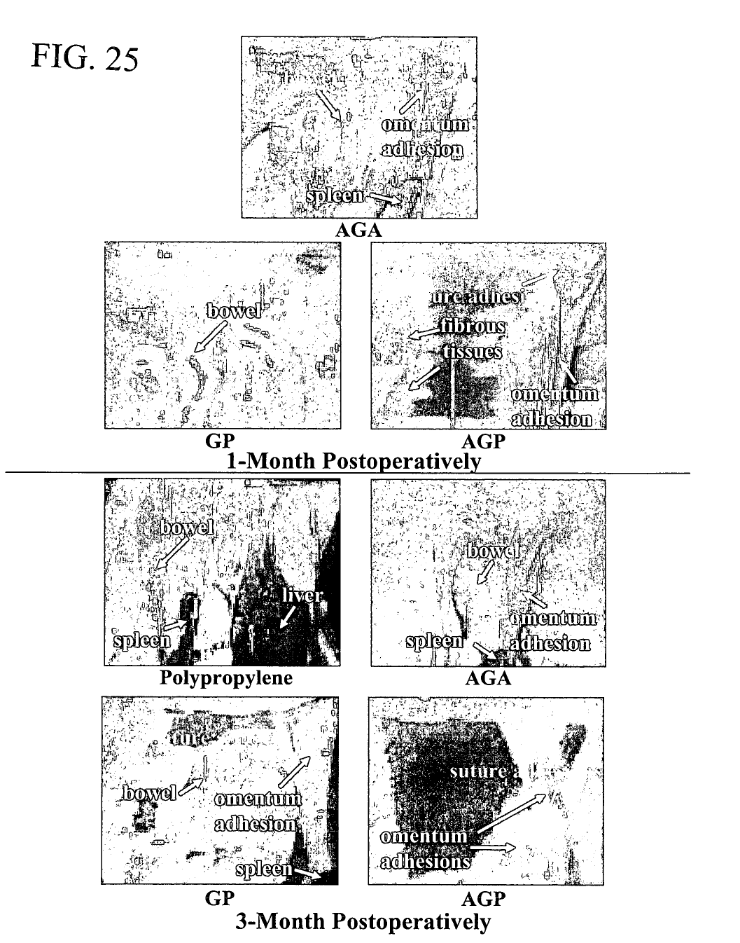

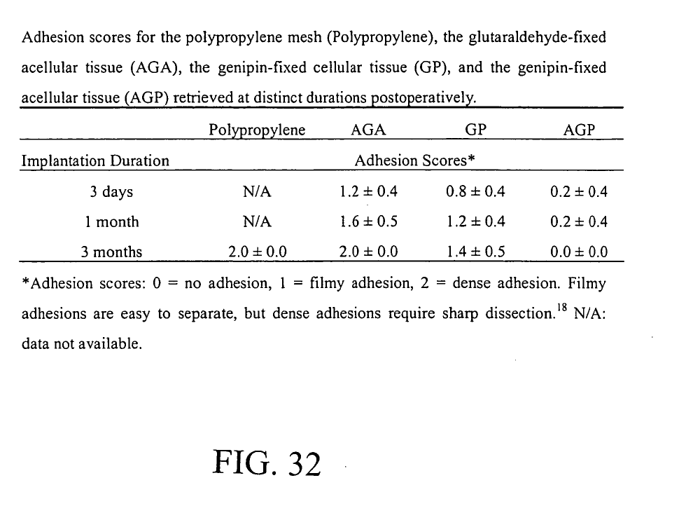

No herniation at the repair site of the abdominal wall was observed for all studied animals throughout the entire course of the study. A table in FIG. 32 shows the adhesion scores for the polypropylene mesh and the AGA, GP, and AGP patches obtained at distinct implantation durations. Representative photographs for each studied group retrieved at 1-month and 3-month postoperatively are presented in FIG. 25. As shown, a filmy to dense adhesion to the visceral organs (bowel, liver, and/or spleen) was observed for the AGA patch retrieved at 3-day and 1-month postoperatively, while a filmy adhesion was seen for the GP patch. In contrast, in four of the animals, the inner surface (visceral side) of the AGP patch was free of any adhesions to the visceral organs. Newly deposited fibrous tissues were loosely organized on the visceral side of the implanted AGP patch. One of the rats had a filmy adhesion to the bowel. However, omentum adhesion to part of the suture was commonly observed for each studied animal.

At retrieval, the abdominal wall was circumferentially incised to the peritoneal cavity to widely expose the repair site. The exposure was performed gently to avoid disturbing any adhesions to viscera or omentum. The appearance of each retrieved sample first was grossly examined and photographed. The formation of adhesions at the prosthesis-visceral peritoneum interface was graded semi-quantitatively from 0˜2: where 0=no adhesion; 1=filmy adhesion; and 2=dense adhesion. Filmy adhesions are easy to separate, but dense adhesions require sharp dissection. Subsequently, a strip dumbbell in shape cut from each retrieved sample was used for the mechanical strength measurement (Sung H W et al., J Biomed Mater Res 1999;47:116). The remainder of the retrieved sample was processed for the histological examination. After the mechanical strength measurement, the same test strip was used for the atomic absorption analysis.

At 3-month postoperatively, dense adhesions to the visceral organs were observed for the polypropylene mesh and the AGA patch, while a filmy to dense adhesion was seen for the GP patch. In contrast, the inner surface of the AGP patch was covered with a glistening mesothelial-like tissue layer. However, omentum adhesion attached along part of the suture line was still observed.

### EXAMPLE 10

### Light Microscopic Examination for Peritoneal Regeneration Study

The samples used for light microscopy were fixed in 10% phosphate buffered formalin for at least 3 days and prepared for histological examination. In the histological examination, the fixed samples were embedded in paraffin and sectioned into a thickness of 5 μm and then stained with hematoxylin and eosin (H&E). The stained sections of each test sample then were examined using light microscopy (Nikon Microphoto-FXA). Additional sections were stained to visualize mesothelial cells as follows (Prophet E B et al., Laboratory Methods in Histotechnology. 2nd ed., Washington: American Registry of Pathology, 1994. pp. 136). Sections were deparaffinized, hydrated, and exposed to a Weigert's hematoxylin working solution for 3 minutes. Subsequently, the sections were extensively rinsed with distilled water, stained with a van Gieson solution for 15 minutes, rinsed again with distilled water, dehydrated through xylene, mounted, and coverslipped. Van Gieson solution is an orthochromatic dye that selectively stains mesothelial cells. Immunohistological staining of macrophages was performed on deparaffinized sections with anti-macrophage-specific F4/80 antibodies (Dako Co., Carpinteria, Calif., USA) and revealed by a peroxidase-antiperoxidase technique. The number of macrophages observed with each studied case was quantified with a computer-based image analysis system (Image-Pro® Plus, Media Cybernetics, Silver Spring, Md., USA). Macrophages were visually identified (original magnification ×800) and the number was counted for each microscopic field. A minimum of five fields was counted for each retrieved sample.

Immunohistochemical staining for neo-collagen type I and III expression in the rat model was performed on paraformaldehyde-fixed slides using rabbit antibodies as the primary. Anti-collagen I and III antibodies (10 μg/mL, Rockland, Gilbertsville, Penn., USA) were incubated for 30 minutes at room temperature, respectively. Secondary antibodies used were Biotin (Vector Laboratories, Burtingame, Calif., USA) conjugated with anti-rabbit antibodies for 30 minutes at room temperature. Detection was done by employing labeled streptavidin-HRP (horseradish peroxidase) for conjugation for 15 minutes. Chromagen DAB (3,3′-diaminobenzidine tetrahydrochloride, Vector Laboratories, Burlingame, Calif., USA) substrates were used for brown color precipitation for 5 min. Specimens were counterstained with hematoxylin (Dako, Carpinteria, Calif., USA) for 5 min and then rinsed in running water for 5 min. The slides were dried at room temperature and covered with mounting media and cover slips.

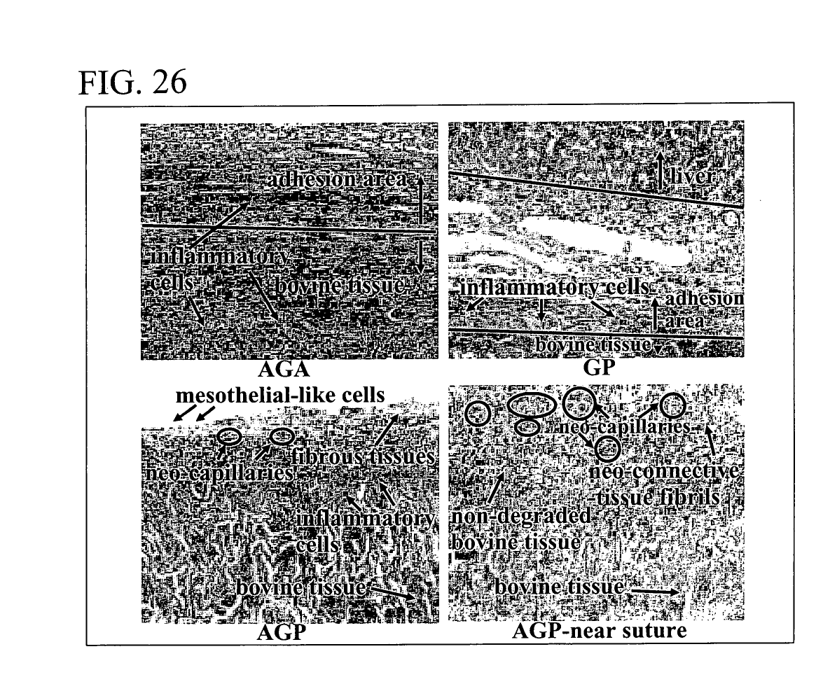

At 3-day postoperatively, inflammatory cells were found mainly surrounding the GP patch (the bovine tissue without cell extraction fixed with genipin). In contrast, inflammatory cells were able to infiltrate into the AGA and AGP patches (the acellular bovine tissues fixed glutaraldehyde or genipin). At 1-month postoperatively, inflammatory cells were still not able to infiltrate into the GP patch, while the depths of inflammatory cells infiltrated into the AGA and AGP patches were greater than their counterparts observed at 3-day postoperatively (FIG. 26). For the AGP patch near the suture line, fibroblasts (migration from the host tissue) and neo-connective-tissue fibrils together with neo-capillaries were clearly observed, indicating that tissue was being regenerated in the AGP patch. Additionally, a layer of mesothelial-like cells was observed on part of the AGP patch. For that not covered with the mesothelial-like cells, fibrous tissue deposition was found. These results indicated that the AGP patch had begun to incorporate into the native abdominal wall tissue. In contrast, no tissue regeneration was observed for the GP and AGA patches.

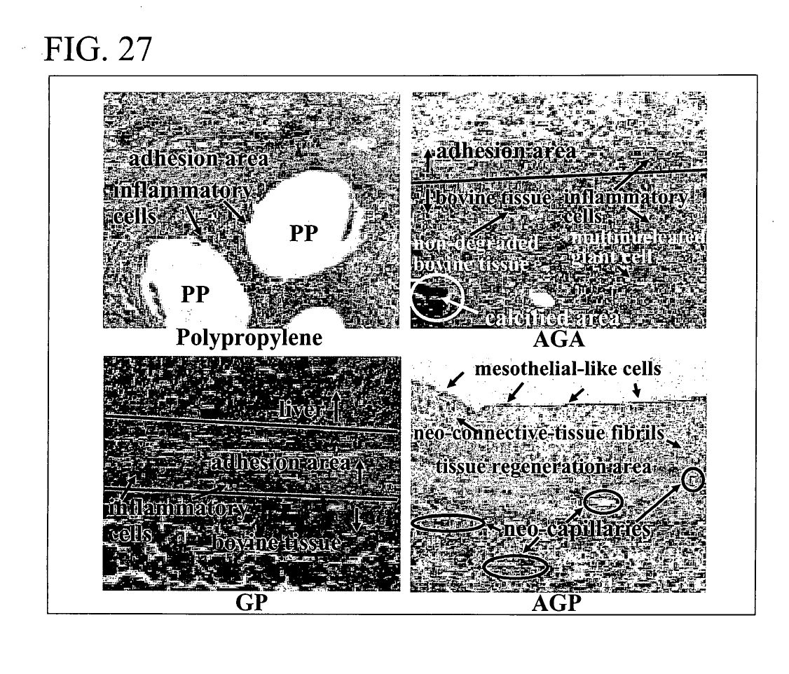

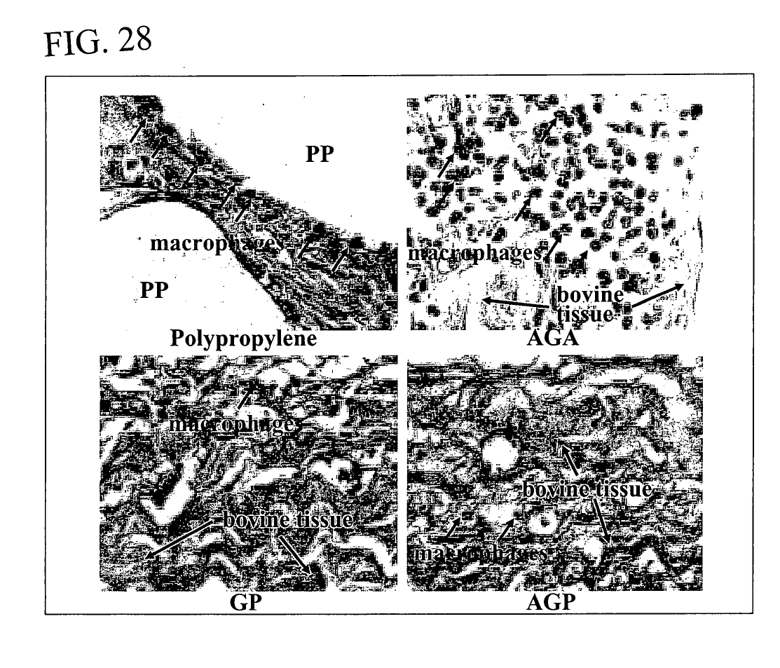

At 3-month postoperatively (FIG. 27), for the polypropylene mesh, inflammatory cells were clearly observed surrounding the knitted polypropylene fibers. There were still a large number of inflammatory cells (macrophages and multinucleated giant cells) observed in the AGA patch and digestion and calcification were observed in its surface layers. Immunohistological staining of macrophages revealed that the degrees of inflammatory reaction for the propylene mesh and the AGA patch were significantly more severe than the GP and AGP patches (FIG. 28, p<0.05). The numbers of macrophages quantified with a computer-based image analysis system were 74±2, 93±8, 4±1, and 7±2 cells per field for the polypropylene mesh and the AGA, GP, and AGP patches, respectively.

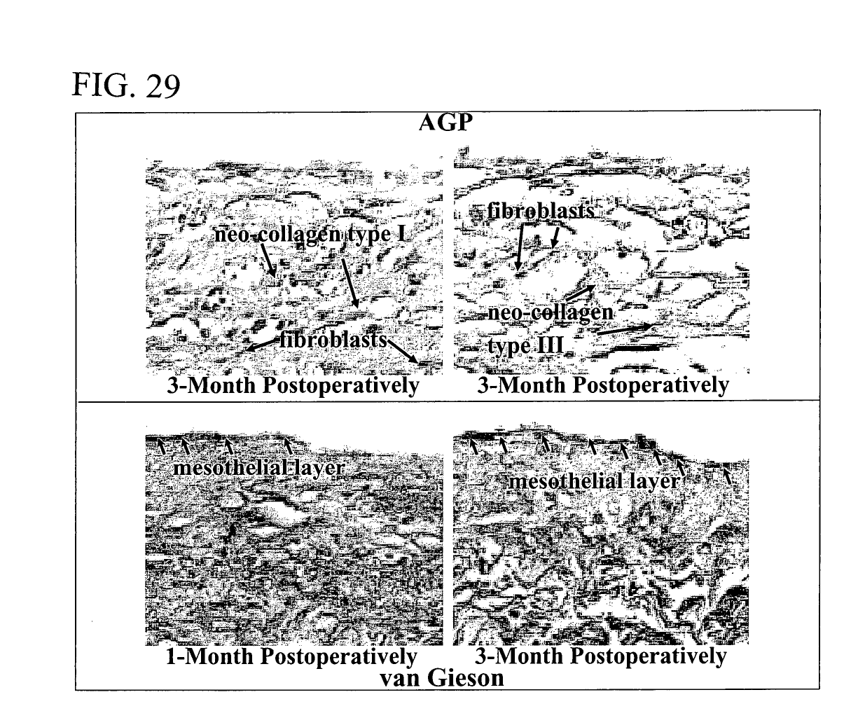

For the GP patch, a denser tissue adhesion formation to its adjacent visceral organs was found (FIG. 27) as compared to its counterpart observed at 1-month postoperatively (FIG. 26). For the AGP patch, the neo-connective-tissue layer was populated with more fibroblasts and was more organized than at 1-month postoperatively. An intact layer of mesothelial-like cells was noted on top of the neo-connective tissues (FIG. 27). The neo-connective tissues were identified by the immunohistochemical stains to contain neo-collagen type I and III fibrils regenerated from the host (rat, FIG. 29). The thin cellular layers observed on the neo-connective tissues for the AGP patch retrieved at 1-month and 3-month postoperatively were further confirmed to be mesothelial cells by the van Gieson stain (FIG. 29).

### EXAMPLE 11

### Cytokine Assay for Peritoneal Regeneration Study

The concentration of IL-1β observed in the peritoneal fluid for each studied group was analyzed using a quantitative sandwich enzyme-linked immunosorbent assay (Bersudsky M et al., Exp Parasitol 2000;94:150). Ninety-six-well plates were coated overnight with primary anti-IL-1β capture monoclonal antibodies (1 μg/ml). The plates were then washed twice with tris/Tween and blocked for 1 hour with PBS/10% BSA (bovine serum albumin) at room temperature. The samples and standards of recombinant IL-1β (Endogen, 15.6 to 1000 pg/ml, Boston, Mass., USA) were then added to the microplates and followed by the addition of biotinylated anti-IL-1β monoclonal antibodies (1:1000 dilution). After incubation for 2 hours at room temperature, the plates were washed three times and further developed by adding Streptavidin-HRP (Endogen, 1:10000 dilution) and TMB-substrate (3,3′,5,5′-tetramethylbenzidine, Endogen). Absorbency at a wavelength of 450 nm was scored in an ELISA reader (Model MRX, Dynatech Laboratories Inc., Chantilly, Va., USA). The amount of IL-1β in samples was extrapolated from a standard curve, consisting of recombinant IL-1β.

Additionally, the concentrations of IL-1β in the peritoneal fluid analyzed by the enzyme-linked immunosorbent assay were: 18.0±2.5 pg/mL for the polypropylene mesh, 23.2±4.0 pg/mL for the AGA patch, 16.7±1.7 pg/mL for the GP patch, and 12.5±1.4 pg/mL for the AGP patch.

### EXAMPLE 12

### Mechanical Strength Determination for Peritoneal Regeneration Study

The mechanical strengths of each studied group before implantation and those retrieved at distinct implantation durations were determined by uniaxial measurements using an Instron material testing machine (Mini 44, Canton, Mass., USA) at a constant speed of 10 mm/min. Fracture was taken to occur when the first decrease in load was detected during extension. Fracture tension was taken as the load at fracture divided by the strip width.

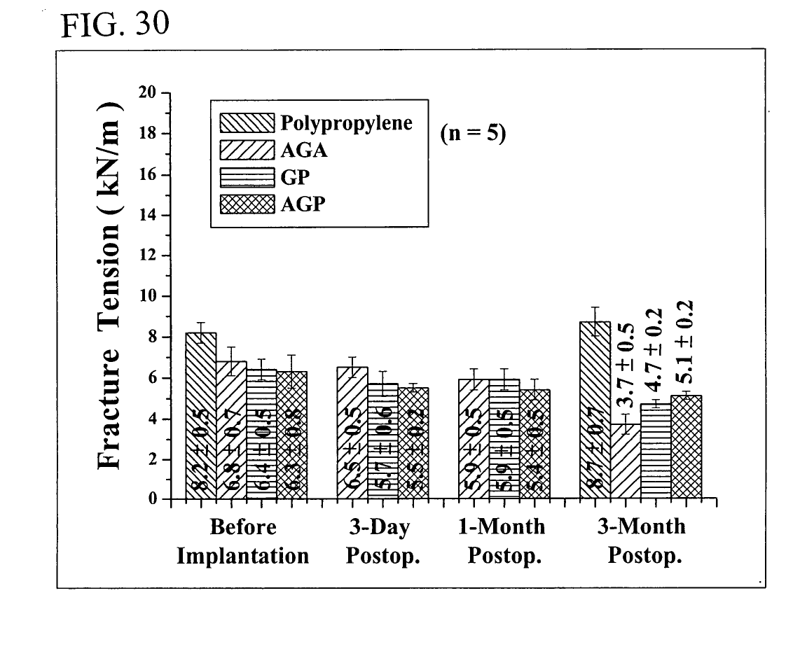

FIG. 30 gives the fracture-tension values of all test samples before implantation and those retrieved at distinct implantation durations. As shown, the fracture-tension value of the polypropylene mesh retrieved at 3-month postoperatively was comparable to that before implantation (p>0.05). In contrast, the fracture-tension values of the GP and AGP patches declined slightly, while that of the AGA patch dropped considerably with increasing the implantation duration (p<0.05).

### EXAMPLE 13

### Atomic Absorption Analysis Peritoneal Regeneration Study

The atomic absorption analysis was employed to determine the calcium content of each retrieved sample. In the analysis, the retrieved samples of each studied group first were lyophilized for 24 hours and weighed. The lyophilized sample then was immersed in a 6N HCl solution (˜3 mg lyophilized tissue per 3 mL 6N HCl) and subsequently hydrolyzed in a microwave hydrolysis system (MDS-2000, CEM Co., Matthews, N.C., USA) for 45 minutes. Finally, the hydrolyzed sample was diluted with a 5% lanthanum chloride in 3N HCl solution. The calcium content of each test sample was determined by an atomic absorption spectrophotometer (Model AA-100, Perkin Elmer Inc., Norwalk, Conn., USA) and was expressed as micrograms per milligram of dry tissue weight.

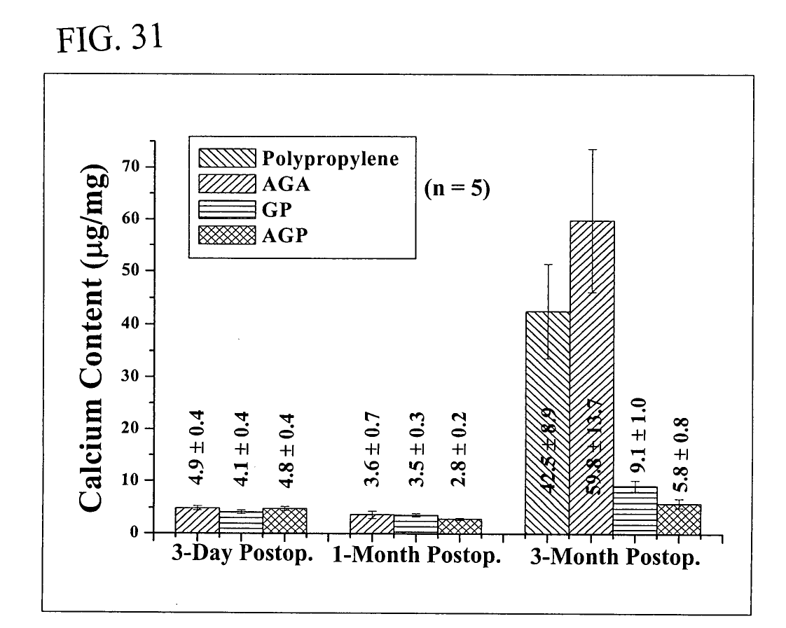

The calcium contents of the polypropylene mesh and the AGA, GP, and AGP patches retrieved at distinct implantation durations, quantified by an atomic absorption spectrophotometer, are presented in FIG. 31. Generally, the calcium contents for the GP and AGP patches were minimal throughout the entire course of the study. On the other hand, the calcium contents for the polypropylene mesh and the AGA patch increased significantly at 3-month postoperatively (p<0.05).

Soon after trauma to the peritoneum, a fibrin matrix forms, which provides the structural framework for normal tissue repair to occur. This normal repair process requires fibrinolysis concurrent with mesothelial repair. The balance of fibrin deposition and degradation seems to be an important determinant in the formation of intra-abdominal adhesions. The importance of fibrin disposition and fibrinolysis in adhesion formation is discussed in detail elsewhere (Jeremy T et al., In: dizerega, G. S., editor. Peritoneal Surgery. 1st ed., N.Y.: Springer, 2000. pp. 133-139). Under ischemic conditions, often associated with surgical injury, the normal fibrinolytic activity of tissue associated with mesothelial repair is compromised, allowing the fibrin matrix to persist and gradually mature into an organized fibrous adhesion.

Various materials have been used in an attempt to reduce the formation of postoperative abdominal adhesion after incisional peritoneal trauma. Polypropylene mesh remains the most widely used implant in the repair of abdominal wall defects and hernias. However, a number of complications were reported clinically when using the knitted polypropylene mesh as a surgical repair material. The present study confirmed its high incidence of adhesion formation reported (FIG. 25 and FIG. 32).

Acellular biological tissues have been proposed to be used as natural biomaterials for soft tissue repair and tissue engineering. Natural biomaterials are composed of extracellular matrix proteins that are conserved among different species and that can serve as scaffolds for cell attachment, migration, and proliferation. The ultrastructures and biochemical properties of acellular bovine pericardia were investigated previously by our group. After cell extraction, light and electron microscopic examinations indicated that all cellular constituents were removed from the bovine pericardium. It left open spaces in the acellular tissue. Biochemical analyses confirmed that the acellular bovine pericardium consisted primarily of insoluble collagen, elastin, and tightly bound glycosaminoglycans. Additionally, the thermal stability (denaturation temperature), mechanical property, and capability against enzymatic degradation of the bovine pericardial tissue remained unaltered after cell extraction.

However, even with complete extraction of cellular proteins, it would still be anticipated a cross-species response directed toward the structural proteins if acellular tissues were used as a xenograft. This cross-species response due to the structural proteins may be further reduced by modifying acellular tissues with a crosslinking agent. In the study, the acellular bovine tissues were fixed with glutaraldehyde (AGA) or genipin (AGP). Using its aldehyde functional groups, glutaraldehyde reacts primarily with the ε-amino groups of lysyl or hydroxylysyl residues within biological tissues. The mechanism of fixation of biological tissues with glutaraldehyde can be found in the literature. Genipin and its related iridoid glucosides extracted from the fruits of Gardenia jasminoides ELLIS have been widely used as an antiphlogistic and cholagogue in herbal medicine. It was found in our previous study that genipin can react with the free amino groups of lysine, hydroxylysine, or arginine residues and form intramolecular and intermolecular crosslinks within biological tissues (Sung HW et al., J Biomed Mater Res 1999;47:116).

In the animal study disclosed above, it was found that inflammatory cells typical of a foreign-body response were present adjacent to the GP patch (made of cellular tissue) and no tissue regeneration was observed throughout the entire course of the study. In contrast, host cells (inflammatory cells, fibroblasts, and neocapillaries) were able to infiltrate into the AGA and AGP patches (made of acellular tissues). Infiltration of host cells into acellular tissues (the AGA and AGP patches) may be caused by the extraction of soluble proteins, lipids, nucleic acids, salts, and carbonhydrates, leading the tissues more permeable to cellular infiltrates. However, the AGA patch elicited a significantly stronger host-tissue response than the AGP patch. The host cells infiltrated into the AGA patch were mostly inflammatory cells (e.g., macrophages and multinucleated giant cells, FIGS. 26-28).

Following surgery, the macrophages increase in number and change function. These postsurgical macrophages are entirely different from the resident macrophages and secrete variable substances, including collagenase, elastase, interleukins (IL) 1 and 6, etc. The immunochemical stain of labeled macrophages revealed that the number of macrophages observed for the AGA patch was significantly greater than the AGP patch (FIG. 28). Additionally, the peritoneal fluid level of IL-1β was significantly higher for the AGA patch than the AGP patch. It is known that the levels of TGF-β1, TNF-α, and IL-1 were higher in surgically induced adhesions in rodents and in humans with adhesions.

Tissue degradation induced by the host inflammatory reaction may reduce the mechanical strengths of the AGA, GP, and AGP patches (FIG. 30). Previous studies have shown that implanted biological tissues provoke a cellular response that leads to physical invasion of the implant by various inflammatory cells such as polymorphonuclear leukocytes, macrophages, and fibroblasts (Chang Y et al., Biomaterials 2002;23:2447). Macrophages are known to be able to secrete collagenase among other proteases. The results obtained at 3-month postoperatively indicated that the mechanical strength of the AGA patch was the lowest among all studied groups, because of its strongest inflammatory reaction observed.

Unlike the AGA patch, the AGP patch retrieved at 1-month postoperatively became well integrated with the host tissue near the suture line, as shown by histology (the observed neo-connective tissues, fibroblasts, and neo-capillaries, FIG. 26). Additionally, there were some neo-mesothelial cells, identified by the van Gieson stain (FIG. 29), observed on the AGP patch. It is known that rapid integration with the host is essential for long-term graft viability. At 3-month postoperatively, a neo-peritoneum was observed on the inner surface of the AGP patch. The neo-peritoneum was homogeneous and composed of organized vascularized connective tissues covered by an intact layer of mesothelial cells (FIGS. 27 and 29).

Omentum adhesion attached along part of the suture line was commonly observed for each studied animal (FIG. 25). Considerable experimental results indicated that peritoneal suturing increased adhesion formation. When the omentum was present, adhesions were far more prevalent if the abdominal wall had been resected. The lack of formation of intra-abdominal adhesions for the AGP patch observed in the study may be due to the regeneration of a neo-mesothelial layer on its peritoneal surface. It is well documented that mesothelial cells prevent adhesions. It was reported that a pure culture of mesothelial cells was able to induce fibrinolysis (Baptista M L et al., J Am Coll Surg 2000;190:271). Another study suggested that the mesothelial fibrinolytic properties are associated with the secretion of tissue plasminogen activator. These results likely explained the observation that once the surface of the AGP patch was populated with mesothelial cells, it remained resistant to adhesion formation.

At 3-month postoperatively, the calcium contents of the polypropylene mesh and the AGA patch increased significantly, while those of the GP and AGP patches stayed minimal (FIG. 31). It was reported in the literature that one of the major problems of biological tissue is calcification. Although calcification of bioprostheses is clearly multifactorial, the exact mechanisms are yet to be elucidated. The observed differences in the aforementioned results between the AGA and AGP patches may be attributed to that the cytotoxicity of genipin is significantly lower than glutaraldehyde (Chang Y et al., J Thorac Cardiovasc Surg 2001;122:1208). Although it is a widely used fixative, glutaraldehyde generally does not allow remodeling of the tissue, generates cytotoxic residuals, and is associated with calcification.

Some aspects of the invention relate to a method of repairing a tissue or organ defect in a patient, comprising (a) providing an acellular tissue sheet material having mechanical strengths; (b) repairing the defect by appropriately placing the tissue material at the defect; and (c) allowing tissue regeneration into the tissue material. By way of illustration, the tissue sheet material may be placed at the defect site by suturing, stapling, connecting, or welding to the defect. Other means for placing the tissue sheet material to repair the defect is within the scope of the present invention. In one embodiment, the defect is an abdominal wall defect, a vascular wall defect, a valvular leaflet defect, or a heart tissue defect. In another embodiment, the tissue sheet material further comprises at least one growth factor selected from a group consisting of vascular endothelial growth factor, transforming growth factor-beta, insulin-like growth factor, platelet derived growth factor, fibroblast growth factor, and combination thereof. In still another embodiment, the tissue sheet material further comprises ginsenoside Rg₁, ginsenoside Re, at least one bioactive agent.

Some aspects of the invention relate to a method of treating postsurgical tissue or organ adhesion comprising: (a) providing an acellular tissue sheet material; (b) placing the acellular tissue sheet material around, about, or adjacent to the tissue or organ to be treated; and (c) preventing the tissue sheet material from forming the postsurgical adhesion by establishing a anti-adhesion barrier. In a further embodiment, the adhesion is abdominal adhesion. In another further embodiment, the tissue sheet material is crosslinked with a crosslinking agent or with ultraviolet irradiation.

Some aspects of the invention relate to a method of treating postsurgical tissue or organ adhesion comprising topically administering an anti-adhesion solution at about the tissue or organ of the surgical site, wherein the solution comprises a crosslinkable biological solution and a crosslinking agent. In a further embodiment, the crosslinking agent is with minimal cytotoxicity and is selected from a group consisting of genipin, its analog, derivatives, and combination thereof, aglycon geniposidic acid, epoxy compounds, dialdehyde starch, glutaraldehyde, formaldehyde, dimethyl suberimidate, carbodiimides, succinimidyls, diisocyanates, acyl azide, reuterin, and combination thereof. In a further embodiment, the anti-adhesion solution further comprises at least one growth factor selected from a group consisting of vascular endothelial growth factor, transforming growth factor-beta, insulin-like growth factor, platelet derived growth factor, fibroblast growth factor, ginsenoside Rg₁, growth factor and ginsenoside Re growth factor.

Genipin Crosslinked Drug Carriers

It is one object of the present invention to provide a drug-collagen-genipin and/or drug-chitosan-genipin compound that is loadable onto an implant/stent or deliverable to a target tissue enabling drug slow-release to the target tissue. In one preferred embodiment, the compound is loaded onto the outer periphery of the stent enabling drug slow-release to the surrounding tissue.

The drugs used in the current generation drug eluting cardiovascular stents include two major mechanisms: cytotoxic and cytostatic. Some aspects of the invention relating to the drugs used in collagen-drug-genipin compound from the category of cytotoxic mechanism comprise actinomycin D, paclitaxel, vincristin, methotrexate, and angiopeptin. Some aspects of the invention relating to the drugs used in collagen-drug-genipin compound from the category of cytostatic mechanism comprise batimastat, halofuginone, sirolimus, tacrolimus, everolimus, tranilast, dexamethasone, and mycophenolic acid (MPA). Some aspects of the present invention provide a bioactive agent in a bioactive agent-eluting device, wherein the bioactive agent is selected from a group consisting of actinomycin D, paclitaxel, vincristin, methotrexate, and angiopeptin, batimastat, halofuginone, sirolimus, tacrolimus, everolimus, tranilast, dexamethasone, and mycophenolic acid.

Everolimus with molecular weight of 958 (a chemical formula of C₅₃H₈₃NO₁₄) is poorly soluble in water and is a novel proliferation inhibitor. There is no clear upper therapeutic limit of everolimus. However, thrombocytopenia occurs at a rate of 17% at everolimus trough serum concentrations above 7.8 ng/ml in renal transplant recipients (Expert Opin Investig Drugs 2002;11(12):1845-1857). In a patient, everolimus binds to cytosolic immunophyllin FKBP12 to inhibit growth factor-driven cell proliferation. Everolimus has shown promising results in animal studies, demonstrating a 50% reduction of neointimal proliferation compared with a control bare metal stent.

Straub et al. in U.S. Pat. No. 6,395,300 discloses a wide variety of drugs that are useful in the methods and compositions described herein, entire contents of which, including a variety of drugs, are incorporated herein by reference. Drugs contemplated for use in the compositions described in U.S. Pat. No. 6,395,300 and herein disclosed include the following categories and examples of drugs and alternative forms of these drugs such as alternative salt forms, free acid forms, free base forms, and hydrates:
analgesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, and meprobamate);
antiasthamatics (e.g., ketotifen and traxanox);
antibiotics (e.g., neomycin, streptomycin, chloramphenicol, cephalosporin, ampicillin, penicillin, tetracycline, and ciprofloxacin);
antidepressants (e.g., nefopam, oxypertine, doxepin, amoxapine, trazodone, amitriptyline, maprotiline, phenelzine, desipramine, nortriptyline, tranylcypromine, fluoxetine, doxepin, imipramine, imipramine pamoate, isocarboxazid, trimipramine, and protriptyline);
antidiabetics (e.g., biguanides and sulfonylurea derivatives);
antifungal agents (e.g., griseofulvin, ketoconazole, itraconizole, amphotericin B, nystatin, and candicidin);
antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine);
anti-inflammatories (e.g., (non-steroidal) indomethacin, ketoprofen, flurbiprofen, naproxen, ibuprofen, ramifenazone, piroxicam, (steroidal) cortisone, dexamethasone, fluazacort, celecoxib, rofecoxib, hydrocortisone, prednisolone, and prednisone);
antineoplastics (e.g., cyclophosphamide, actinomycin, bleomycin, daunorubicin, doxorubicin hydrochloride, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof, vinblastine, vincristine, tamoxifen, piposulfan,);
antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene);
immunosuppressive agents (e.g., cyclosporine, azathioprine, mizoribine, and FK506 (tacrolimus));
antimigraine agents (e.g., ergotamine, propanolol, isometheptene mucate, and dichloralphenazone);
sedatives/hypnotics (e.g., barbiturates such as pentobarbital, pentobarbital, and secobarbital; and benzodiazapines such as flurazepam hydrochloride, triazolam, and midazolam);
antianginal agents (e.g., beta-adrenergic blockers; calcium channel blockers such as nifedipine, and diltiazem; and nitrates such as nitroglycerin, isosorbide dinitrate, pentaerythritol tetranitrate, and erythrityl tetranitrate);
antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, and prochlorperazine);
antimanic agents (e.g., lithium carbonate);
antiarrhythmics (e.g., bretylium tosylate, esmolol, verapamil, amiodarone, encainide, digoxin, digitoxin, mexiletine, disopyramide phosphate, procainamide, quinidine sulfate, quinidine gluconate, quinidine polygalacturonate, flecainide acetate, tocainide, and lidocaine);
antiarthritic agents (e.g., phenylbutazone, sulindac, penicillanine, salsalate, piroxicam, azathioprine, indomethacin, meclofenamate, gold sodium thiomalate, ketoprofen, auranofin, aurothioglucose, and tolmetin sodium);
antigout agents (e.g., colchicine, and allopurinol);
anticoagulants (e.g., heparin, heparin sodium, and warfarin sodium);
thrombolytic agents (e.g., urokinase, streptokinase, and alteplase);
antifibrinolytic agents (e.g., aminocaproic acid);
hemorheologic agents (e.g., pentoxifylline);
antiplatelet agents (e.g., aspirin);
anticonvulsants (e.g., valproic acid, divalproex sodium, phenytoin, phenytoin sodium, clonazepam, primidone, phenobarbitol, carbamazepine, amobarbital sodium, methsuximide, metharbital, mephobarbital, mephenytoin, phensuximide, paramethadione, ethotoin, phenacemide, secobarbitol sodium, clorazepate dipotassium, and trimethadione);
antiparkinson agents (e.g., ethosuximide);
antihistamines/antipruritics (e.g., hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, and methdilazine);
agents useful for calcium regulation (e.g., calcitonin, and parathyroid hormone);
antibacterial agents (e.g., amikacin sulfate, aztreonam, chloramphenicol, chloramphenicol palirtate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate);
antiviral agents (e.g., interferon alpha, beta or gamma, zidovudine, amantadine hydrochloride, ribavirin, and acyclovir);
antimicrobials (e.g., cephalosporins such as cefazolin sodium, cephradine, cefaclor, cephapirin sodium, ceftizoxime sodium, cefoperazone sodium, cefotetan disodium, cefuroxime azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium; penicillins such as ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, and nafcillin sodium; erythromycins such as erythromycin ethylsuccinate, erythromycin, erythromycin estolate, erythromycin lactobionate, erythromycin stearate, and erythromycin ethylsuccinate; and tetracyclines such as tetracycline hydrochloride, doxycycline hyclate, and minocycline hydrochloride, azithromycin, clarithromycin);
anti-infectives (e.g., GM-CSF);
bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate; anticholinergic agents such as ipratropium bromide; xanthines such as aminophylline, dyphylline, metaproterenol sulfate, and aminophylline; mast cell stabilizers such as cromolyn sodium; inhalant corticosteroids such as beclomethasone dipropionate (BDP), and beclomethasone dipropionate monohydrate; salbutamol; ipratropium bromide; budesonide; ketotifen; salmeterol; xinafoate; terbutaline sulfate; triamcinolone; theophylline; nedocromil sodium; metaproterenol sulfate; albuterol; flunisolide; fluticasone proprionate;
steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium);
hypoglycemic agents (e.g., human insulin, purified beef insulin, purified pork insulin, glyburide, chlorpropamide, glipizide, tolbutarnide, and tolazamide);
hypolipidemic agents (e.g., clofibrate, dextrothyroxine sodium, probucol, pravastitin, atorvastatin, lovastatin, and niacin);
proteins (e.g., DNase, alginase, superoxide dismutase, and lipase);
nucleic acids (e.g., sense or anti-sense nucleic acids encoding any therapeutically useful protein, including any of the proteins described herein);
agents useful for erythropoiesis stimulation (e.g., erythropoietin);
antiulcer/antireflux agents (e.g., famotidine, cimetidine, and ranitidine hydrochloride);
antinauseants/antiemetics (e.g., meclizine hydrochloride, nabilone, prochlorperazine, dimenhydrinate, promethazine hydrochloride, thiethylperazine, and scopolamine);
as well as other drugs useful in the compositions and methods described herein include mitotane, halonitrosoureas, anthrocyclines, ellipticine, ceftriaxone, ketoconazole, ceftazidime, oxaprozin, albuterol, valacyclovir, urofollitropin, famciclovir, flutamide, enalapril, mefformin, itraconazole, buspirone, gabapentin, fosinopril, tramadol, acarbose, lorazepan, follitropin, glipizide, omeprazole, fluoxetine, lisinopril, tramsdol, levofloxacin, zafirlukast, interferon, growth hormone, interleukin, erythropoietin, granulocyte stimulating factor, nizatidine, bupropion, perindopril, erbumine, adenosine, alendronate, alprostadil, benazepril, betaxolol, bleomycin sulfate, dexfenfluramine, diltiazem, fentanyl, flecainid, gemcitabine, glatiramer acetate, granisetron, lamivudine, mangafodipir trisodium, mesalamine, metoprolol fumarate, metronidazole, miglitol, moexipril, monteleukast, octreotide acetate, olopatadine, paricalcitol, somatropin, sumatriptan succinate, tacrine, verapamil, nabumetone, trovafloxacin, dolasetron, zidovudine, finasteride, tobramycin, isradipine, tolcapone, enoxaparin, fluconazole, lansoprazole, terbinafine, pamidronate, didanosine, diclofenac, cisapride, venlafaxine, troglitazone, fluvastatin, losartan, imiglucerase, donepezil, olanzapine, valsartan, fexofenadine, calcitonin, and ipratropium bromide. These drugs are generally considered to be water soluble.

Preferred drugs useful in the present invention may include albuterol, adapalene, doxazosin mesylate, mometasone furoate, ursodiol, amphotericin, enalapril maleate, felodipine, nefazodone hydrochloride, valrubicin, albendazole, conjugated estrogens, medroxyprogesterone acetate, nicardipine hydrochloride, zolpidem tartrate, amlodipine besylate, ethinyl estradiol, omeprazole, rubitecan, amlodipine besylate/benazepril hydrochloride, etodolac, paroxetine hydrochloride, paclitaxel, atovaquone, felodipine, podofilox, paricalcitol, betamethasone dipropionate, fentanyl, pramipexole dihydrochloride, Vitamin D₃ and related analogues, finasteride, quetiapine fumarate, alprostadil, candesartan, cilexetil, fluconazole, ritonavir, busulfan, carbamazepine, flumazenil, risperidone, carbemazepine, carbidopa, levodopa, ganciclovir, saquinavir, amprenavir, carboplatin, glyburide, sertraline hydrochloride, rofecoxib carvedilol, clobustasol, diflucortolone, halobetasolproprionate, sildenafil citrate, celecoxib, chlorthalidone, imiquimod, simvastatin, citalopram, ciprofloxacin, irinotecan hydrochloride, sparfloxacin, efavirenz, cisapride monohydrate, lansoprazole, tamsulosin hydrochloride, mofafinil, clarithromycin, letrozole, terbinafine hydrochloride, rosiglitazone maleate, diclofenac sodium, lomefloxacin hydrochloride, tirofiban hydrochloride, telmisartan, diazapam, loratadine, toremifene citrate, thalidomide, dinoprostone, mefloquine hydrochloride, trandolapril, docetaxel, mitoxantrone hydrochloride, tretinoin, etodolac, triamcinolone acetate, estradiol, ursodiol, nelfinavir mesylate, indinavir, beclomethasone dipropionate, oxaprozin, flutamide, famotidine, nifedipine, prednisone, cefuroxime, lorazepam, digoxin, lovastatin, griseofulvin, naproxen, ibuprofen, isotretinoin, tamoxifen citrate, nimodipine, amiodarone, and alprazolam.

Specific non-limiting examples of some drugs that fall under the above categories include paclitaxel, docetaxel and derivatives, epothilones, nitric oxide release agents, heparin, aspirin, coumadin, PPACK, hirudin, polypeptide from angiostatin and endostatin, methotrexate, 5-fluorouracil, estradiol, P-selectin Glycoprotein ligand-1 , chimera, abciximab, exochelin, eleutherobin and sarcodictyin, fludarabine, sirolimus, tranilast, VEGF, transforming growth factor (TGF)-beta, Insulin-like growth factor (IGF), platelet derived growth factor (PDGF), fibroblast growth factor (FGF), RGD peptide, beta or gamma ray emitter (radioactive) agents, and dexamethasone, tacrolimus, actinomycin-D, batimastat etc.

Sirolimus is a naturally occurring macrolide antibiotic produced by the fungus Streptomyces found in Easter Island. It was discovered by Wyeth-Ayerst in 1974 while screening fermentation products. Sirolimus with molecular weight of 916 (a chemical formula of C₅₁H₇₉NO₁₃) is non-water soluble and is a potential inhibitor of cytokine and growth factor mediated cell proliferation. FDA approved its use as oral immunosuppressive agents with a formulation of 2 to 5 mg/dose. The suggested drug-eluting efficacy is about 140 micrograms/cm², 95% drug release at 90 days and 30% drug-to-polymer ratio.

In some aspect of the present invention, the drug (also referred as a bioactive agent) may broadly comprise, but not limited to, synthetic chemicals, biotechnology-derived molecules, herbs, health food, extracts, and/or alternate medicines; for example, including allicin and its corresponding garlic extract, ginsenosides and the corresponding ginseng extract, flavone/terpene lactone and the corresponding ginkgo biloba extract, glycyrrhetinic acid and the corresponding licorice extract, and polyphenol/proanthocyanides and the corresponding grape seed extract.

Local Atherosclerosis Reducing Agent

It was reported in JAMA. 2003;290:2292-2300 and 2322-2324, entire contents of which are incorporated herein by reference, that infusion of Milano Apoprotein causes rapid regression of atherosclerosis in patients with acute coronary syndromes (ACS), according to the results of a preliminary randomized trial published in the November 5 issue of The Journal of the American Medical Association. This intravenous therapy targeting high-density lipoprotein cholesterol (HDL-C) may represent a new approach to the future treatment of atherosclerosis. “Approximately 40 carriers with a naturally occurring variant of apolipoprotein A-I known as ApoA-I Milano are characterized by very low levels of HDL-C, apparent longevity, and much less atherosclerosis than expected for their HDL-C levels,” write Steven E. Nissen, Md., from the Cleveland Clinic Foundation in Ohio, and colleagues. Of 123 patients with ACS, aged 38 to 82 years, who were screened between November 2001 and March 2003 at 10 U.S. centers, 57 patients were randomized. Of 47 patients who completed the protocol, 11 received placebo, 21 received low-dose and 15 received high-dose recombinant ApoA-I Milano/phospholipid complexes (ETC-216) by intravenous infusion at weekly intervals for five doses. Serial intravascular ultrasound measurements within two weeks of ACS and after treatment revealed that the mean percentage of atheroma volume decreased by 1.06% in the combined ETC-216 group compared with an increase of 0.14% in the placebo group. In the combined treatment groups, the absolute reduction in atheroma volume was a 4.2% decrease from baseline.

This initial trial of an exogenously produced HDL mimetic demonstrated significant evidence of rapid regression of atherosclerosis. The authors write, “the potential utility of the new approach must be fully explored in a larger patient population with longer follow-up, assessing a variety of clinical end points, including morbidity and mortality”. In an accompanying editorial, Daniel J. Rader, MD, from the University of Pennsylvania School of Medicine in Philadelphia, discusses several study limitations, including small sample size, short treatment duration, unclear relationship of intravascular ultrasound findings to clinical benefit, and failure to compare infusion of normal ApoA-I with that of ApoA-I Milano.

The mechanisms of action of ApoA-I Milano and phospholipid complex that result in regression of atherosclerosis are unknown but presumably are related to an increase in reverse cholesterol transport from atheromatous lesions to the serum with subsequent modification and removal by the liver (JAMA. 2003;290:2292-2300). The cysteine substitution for arginine at position 173 for the ApoA-I Milano variant allows dimerization, forming large HDL particles that may be particularly active in reverse cholesterol transport. In vitro experiments have demonstrated increased cholesterol efflux from cholesterol-loaded hepatoma cells incubated with serum from ApoA-I Milano carriers or from transgenic mice. As a result, some day patients with acute coronary syndromes may receive ‘acute induction therapy’ with HDL-based therapies for rapid regression and stabilization of lesions, followed by long-term therapy to prevent the regrowth of these lesions. In this model, long-term HDL-based therapies will still be needed as a vital component of the preventive phase.

The bioactive agent of the present invention further comprises ApoA-I Milano, recombinant ApoA-I Milano/phospholipid complexes (ETC-216), and the like (as atherosclerosis reducing agent). In one embodiment, the atherosclerosis reducing agent is used to treat both stenotic plaque and vulnerable plaque of a patient for regression and stabilization of lesions. Some aspects of the invention relate to a method for promoting atherosclerosis regression comprising: providing crosslinkable biological solution to the target tissue, wherein the crosslinkable biological solution is loaded with at least one atherosclerosis reducing agent. In one embodiment, the at least one atherosclerosis reducing agent comprises ApoA-I Milano or recombinant ApoA-I Milano/phospholipid complexes.

While the preventive and treatment properties of the foregoing therapeutic substances, agents, drugs, or bioactive agents are well known to those having ordinary skill in the art, the substances or agents are provided by way of example and are not meant to be limiting. Other therapeutic substances are equally applicable for use with the disclosed methods, devices, and compositions.

It is another object of the present invention to provide a crosslinkable biological solution kit comprising a first readily mixable crosslinkable biological solution component and a second crosslinker component, wherein an operator can add appropriate drug or bioactive agent to the kit and obtain a drug-collagen-genipin and/or drug-chitosan-genipin compound that is loadable onto an implant/stent or deliverable to a target tissue enabling drug slow-release to the target tissue. In a further embodiment, the crosslinkable biological solution kit is packaged in a form for topical administration, for percutaneous injection, for intravenous injection, for intramuscular injection, for loading on an implant or biological tissue material, and/or for oral administration.

Some aspects of the invention relate to a method for promoting angiogenesis comprising administering ginsenoside Rg₁ and/or ginsenoside Re onto tissue after radiation therapy to promote neovascularization. Some further aspects of the invention relate to a method for promoting angiogenesis comprising administering ginsenoside Rg₁ and/or ginsenoside Re onto tissue of ulcer or diabetes to promote neovascularization.

From the foregoing description, it should now be appreciated that a novel and unobvious acellular bovine pericardium fixed with genipin (the AGP patch) effectively repairing abdominal wall defects in rats and successfully preventing the formation of postsurgical abdominal adhesions has been disclosed for tissue engineering applications. While the invention has been described with reference to a specific embodiment, the description is illustrative of the invention and is not to be construed as limiting the invention. Various modifications and applications may occur to those who are skilled in the art, without departing from the true spirit and scope of the invention.

1. A method of repairing a tissue or organ defect in a patient, comprising
providing an acellular tissue sheet material having mechanical strengths;
repairing the defect by appropriately placing said tissue material at the defect; and
allowing tissue regeneration into said tissue material.

2. The method of claim 1, wherein the tissue sheet material is selected from a group consisting of a bovine pericardium, an equine pericardium, an ovine pericardium, a porcine pericardium, and a valvular leaflet.

3. The method of claim 1, wherein the tissue sheet material is crosslinked with a crosslinking agent or with ultraviolet irradiation.

4. The method of claim 1, wherein the tissue sheet material is crosslinked with a crosslinking agent selected from a group consisting of genipin, its analog, derivatives, and combination thereof, aglycon geniposidic acid, epoxy compounds, dialdehyde starch, glutaraldehyde, formaldehyde, dimethyl suberimidate, carbodiimides, succinimidyls, diisocyanates, acyl azide, reuterin, and combination thereof.

5. The method of claim 1, wherein the method further comprises a process of increasing porosity of the acellular tissue sheet material, said process being selected from a group consisting of an enzyme treatment process, an acid treatment process, and a base treatment process.

6. The method of claim 5, wherein said increase of porosity of the tissue material is 5% or higher.

7. The method of claim 1, wherein the defect is an abdominal wall defect.

8. The method of claim 1, wherein the defect is a vascular wall defect.

9. The method of claim 1, wherein the defect is a valvular leaflet defect.

10. The method of claim 1, wherein the defect is a heart tissue defect.

11. The method of claim 1, wherein the tissue material further comprises at least one growth factor selected from a group consisting of vascular endothelial growth factor, transforming growth factor-beta, insulin-like growth factor, platelet derived growth factor, fibroblast growth factor, and combination thereof.

12. The method of claim 1, wherein said tissue material further comprises ginsenoside Rg₁ or ginsenoside Re.

13. The method of claim 1, wherein said tissue material further comprises at least one bioactive agent.

14. A method of treating postsurgical tissue or organ adhesion comprising:
providing an acellular tissue sheet material;
placing said acellular tissue sheet material about the tissue or organ to be treated; and
preventing said tissue sheet material from forming the postsurgical adhesion.

15. The method of claim 14, wherein the adhesion is abdominal adhesion.

16. The method of claim 14, wherein the tissue sheet material is crosslinked with a crosslinking agent or with ultraviolet irradiation.

17. The method of claim 14, wherein the tissue sheet material is crosslinked with a crosslinking agent selected from a group consisting of genipin, its analog, derivatives, and combination thereof, aglycon geniposidic acid, epoxy compounds, dialdehyde starch, glutaraldehyde, formaldehyde, dimethyl suberimidate, carbodiimides, succinimidyls, diisocyanates, acyl azide, reuterin, and combination thereof.

18. A method of treating postsurgical tissue or organ adhesion comprising topically administering an anti-adhesion solution at about said tissue or organ of the surgical site, wherein said anti-adhesion solution comprises a crosslinkable biological solution and a crosslinking agent.

19. The method of claim 18, wherein the crosslinking agent is selected from a group consisting of genipin, its analog, derivatives, and combination thereof, aglycon geniposidic acid, epoxy compounds, dialdehyde starch, glutaraldehyde, formaldehyde, dimethyl suberimidate, carbodiimides, succinimidyls, diisocyanates, acyl azide, reuterin, and combination thereof.

20. The method of claim 1, wherein the anti-adhesion solution further comprises at least one growth factor selected from a group consisting of vascular endothelial growth factor, transforming growth factor-beta, insulin-like growth factor, platelet derived growth factor, fibroblast growth factor, ginsenoside Rg₁ growth factor and ginsenoside Re growth factor.
